(19) United States
(12) Patent Application Publication
(10) Pub. No.: US 2018/0236106 A1
(43) Pub. Date: Aug. 23, 2018

(54) RADIOGRAPHIC NANOPARTICLE CONTRAST AGENTS FOR DUAL-ENERGY X-RAY IMAGING AND COMPUTED TOMOGRAPHY SCANNING AND METHODS OF USING THEREOF

(71) Applicant: The Trustees of the University of Pennsylvania, Philadelphia, PA (US)

(72) Inventors: David Peter Cormode, Philadelphia, PA (US); Andrew Maidment, Villanova, PA (US); Pratap Chandra Naha, Philadelphia, PA (US); Roshan Karunamuni, San Diego, CA (US)

(73) Assignee: The Trustees of the University of Pennsylvania, Philadelphia, PA (US)

(21) Appl. No.: 15/752,308

(22) PCT Filed: Aug. 12, 2016
(86) PCT No.: PCT/US2016/046811

§ 371 Date: Feb. 13, 2018

Related U.S. Application Data
(60) Provisional application No. 62/205,154, filed on Aug. 14, 2015.

(51) Int. Cl. A61K 49/00; A61K 49/04

(52) U.S. Cl. CPC A61K 49/0002; A61K 49/0428

(57) ABSTRACT

Nanoparticles and nanoprobes for use as a contrast agent for X-ray imaging techniques, CT scanning techniques, MRI and optical imaging are disclosed. The nanoparticles and nanoprobes include a core having a contrast element characterized by a K-edge value ranging from about 17 to about 49 keV, and a stabilizing element which minimizes one or both of cytotoxicity and immunoreactivity of the contrast element. A first coating layer encapsulates the core, the first coating layer configured to render the nanoparticles soluble in a biological medium. A method for dual energy x-ray imaging includes the steps of administering to a subject the nanoparticles disclosed herein as a contrast agent; acquiring an image with a low energy spectrum; and acquiring an image with a high energy spectrum.

### CROSS REFERENCE TO RELATED APPLICATIONS

This application claims priority to U.S. Provisional Application No. 62/205,154, entitled RADIOGRAPHIC NANOPARTICLE CONTRACT AGENTS FOR DUAL-ENERGY X-RAY IMAGING AND METHODS OF USING THEREOF, filed Aug. 14, 2015, the contents of which are incorporated by reference herein in their entirety.

### STATEMENT OF GOVERNMENT INTEREST

This invention was made with Government support under W81XWH-09-1-0055 and W81XWH-11-1-0246, awarded by the Federal Department of Defense, as well as R03-CA171661, awarded by the National Institutes of Health. The Government has certain rights in the invention.

### FIELD OF THE INVENTION

This invention relates to the field of contrast agents and, more particularly, nanoparticle contrast agents for x-ray imaging and methods for x-ray imaging of tissue using nanoparticle contrast agents.

### BACKGROUND OF THE INVENTION

Breast cancer is the most common form of cancer to affect women and is the second most deadly cancer in women. One of the keys to successful treatment of breast cancer and long-term survival is early detection. Population wide mammography screening programs have been shown to reduce mortality due to breast cancer by at least 15%. However, it is now recognized that mammography is not effective for women with dense breasts (sensitivity of only 30-60%). In addition, women with dense breasts have a 3.25-fold higher risk of breast cancer, independent of issues related to screening. These women are therefore both at high risk of developing breast cancer and have a low likelihood of early detection. While contrast-enhanced MRI can be used for detection of breast cancer in women with dense breasts, the cost of MRI renders it impractical as a population-wide screening tool. New methods that can detect tumors in women with dense breasts early would save lives and reduce costs associated with this disease.

Dual energy (DE) mammography has recently emerged as a clinical tool for breast cancer screening. DE mammography aims to distinguish tumors from adipose/glandular tissue via the different attenuation of materials at different x-ray energies. Contrast agents are used with DE mammography in an attempt to highlight tumors. In this technique, two mammograms are acquired in rapid succession using two different x-ray tube voltages and beam filters, i.e., a high energy (“HE”) and a low energy (“LE”) acquisition. A logarithmically weighted image subtraction is done to create a DE image. The signal from the breast tissue is suppressed and that from the contrast agent is enhanced. A contrast agent that is taken up by an aggressive tumor can therefore improve the conspicuity of lesions.

DE mammography is currently performed using iodinated small molecule contrast agents. However, these iodinated agents have a number of drawbacks, such as patient hypersensitivity, contra-indication in patients with renal insufficiency, very short circulation half-lives and a lack of tumor accumulation.

### SUMMARY OF THE INVENTION

Aspects of the invention relate to nanoparticles for use as a contrast agent, kits for contrast imaging, as well as methods for dual energy x-ray imaging.

In accordance with one aspect, the invention provides nanoparticles for use as a contrast agent for medical imaging techniques. The nanoparticles include a core having a contrast element, wherein the contrast element has a K-edge value ranging from about 17 to about 49 keV and a stabilizing element which minimizes one or both of cytotoxicity and immunoreactivity of the contrast element. A first coating layer encapsulates the core, the first coating layer configured to render the nanoparticles soluble in a biological medium.

In accordance with another aspect, the invention provides a kit for medical imaging. The kit includes a dry-powder dosage formulation including a contrast agent comprising a plurality of nanoparticles, the plurality of nanoparticles each having a core. The core includes a stabilizing element selected from one or more of B, C, N, O, F, Al, Si, P, S, Cl, Ti, V, Cr, Mn, Fe, Co, Ni, Cu, Zn, Ga, Ge, As, Se, Br, Y, Zr, Nb, Mo, Ru, Rh, Pd, Ag, Cd, In, Sn, Sb, Te, I, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Bi, or others. The core further includes a contrast element having a K-edge value ranging from about 17 to about 49 keV. A first coating layer encapsulates the core, the first coating layer configured to render the nanoparticles soluble in a biological medium. The kit also includes a pharmaceutically acceptable carrier solution suitable for injection for reconstituting the dosage formulation.

In accordance with another aspect, the invention provides a method for dual energy x-ray imaging. The method includes the steps of administering to a subject the nanoparticles described herein as a contrast agent; acquiring an image with a low energy spectrum; and acquiring an image with a high energy spectrum.

It is to be understood that both the foregoing general description and the following detailed description are exemplary, but are not restrictive, of the invention.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following Figures:

FIG. 1 is a schematic illustration of the in vitro degradation of a silver nanoparticle contrast agent as known in the prior art;

FIG. 2A is a schematic representation of a nanoparticle contrast agent in accordance with aspects of the present invention;

FIG. 2B is a schematic representation of a nanoparticle contrast agent in accordance with aspects of the present invention;

FIG. 3 is a schematic illustration of the in vitro degradation of a nanoparticle contrast agent in accordance with aspects of the present invention;

FIG. 4 is a flow diagram depicting selected steps of a method for dual energy x-ray imaging in accordance with aspects of the present invention;

FIG. 5 is a schematic representation of a process for synthesizing a nanoparticle contrast agent in accordance with aspects of the present invention;

FIG. 6A is a schematic representation of a process for synthesizing a nanoparticle contrast agent in accordance with aspects of the present invention;

FIG. 6B is a collection of TEM images of gold-silver alloy nanoparticles in accordance with aspects of the present invention;

FIG. 6C is an energy dispersive X-ray spectrum of GSAN formulations in accordance with aspects of the present invention;

FIG. 6D is a photograph of citrate coated and PEGylated GSAN incubated in PBS for one hour in accordance with aspects of the present invention;

FIG. 6E is a photograph of citrate coated and PEGylated GSAN incubated in PBS for 24 hours in accordance with aspects of the present invention;

FIG. 7A is a listing of chemical structures of ligands for use with nanoparticle contrast agents in accordance with aspects of the present invention;

FIG. 7B is a TEM image of gold-silver alloy nanoparticles in accordance with aspects of the present invention;

FIG. 7C is a TEM image of gold-silver alloy nanoparticles in accordance with aspects of the present invention;

FIG. 7D is a photograph of 100, 90, 80, 70, 60 and 50% Ag gold-silver alloy nanoparticles in accordance with aspects of the present invention;

FIG. 7E is a graph depicting the viability of cells incubated with various nanoparticles in accordance with aspects of the present invention;

FIG. 7F is a graph depicting UV-visible spectra of GSAN formulations in accordance with aspects of the present invention;

FIG. 8A is a graph depicting silver ion release from different formulations in deionized (DI) water in accordance with aspects of the present invention;

FIG. 8B is a graph depicting silver ion release from different formulations in simulated lysosomal fluid in accordance with aspects of the present invention;

FIG. 8C is a graph depicting the viability of cells incubated with various nanoparticles in accordance with aspects of the present invention;

FIG. 8D is a graph depicting the viability of cells incubated with various nanoparticles in accordance with aspects of the present invention;

FIG. 9A is a graph depicting reactive oxygen species generation in J774A.1 cells in accordance with aspects of the present invention;

FIG. 9B is a graph depicting reactive oxygen species generation in HepG2 cells in accordance with aspects of the present invention;

FIG. 9C is a graph depicting DNA damage effects of GSAN to J774A.1 cells in accordance with aspects of the present invention;

FIG. 9D is a graph depicting DNA damage effects of GSAN to HepG2 cells in accordance with aspects of the present invention;

FIG. 10A is a collection of dual energy mammography phantom images in accordance with aspects of the present invention;

FIG. 10B is a collection of dual energy mammography phantom images in accordance with aspects of the present invention;

FIG. 10C is a graph depicting the quantification of DE mammography phantom data in accordance with aspects of the present invention;

FIG. 10D is a collection of CT phantom images of silver nitrate, iopamidol, and GSAN (Ag-80) scanned at 120 kV in accordance with aspects of the present invention;

FIG. 10E is a graph depicting CT attenuation rates of different agents accordance with aspects of the present invention;

FIG. 11A is a DE mammography image of a mouse without a tumor, injected with GSAN in accordance with aspects of the present invention;

FIG. 11B is a DE mammography image of representative tumor-bearing mice pre-injection and at 30 minutes post-injection with iopamidol or GSAN in accordance with aspects of the present invention;

FIG. 11C is a graph depicting SNR in the tumors of FIG. 11B at different time points in accordance with aspects of the present invention;

FIG. 11D is a collection of dual energy mammography phantom images in accordance with aspects of the present invention;

FIG. 11E is a computed tomography image in accordance with aspects of the present invention;

FIG. 12A is a collection of 3D volume rendered CT images of a mouse without a tumor, injected with GSAN in accordance with aspects of the present invention; hearts, kidneys, and intestines are labeled H, K, and I, respectively, and green circles indicate the bladder;

FIG. 12B is a collection of 2D CT images of a tumor-bearing mouse showing accumulation of GSAN in the tumors at different time points; yellow circles indicate tumors;

FIG. 12C is a graph depicting the quantification of CT attenuation in different organs of mice injected with GSAN at different time points in accordance with aspects of the present invention;

FIG. 13 is a graph depicting the biodistribution of Ag-60 GSAN in different organs at 2 hours post-injection in accordance with aspects of the present invention;

FIG. 14A is a TEM image of nanoparticles in accordance with aspects of the present invention;

FIG. 14B is an EDS image of nanoparticles in accordance with aspects of the present invention;

FIG. 14C is a TEM image of nanoparticles in accordance with aspects of the present invention;

FIG. 14D is a TEM image of nanoparticles in accordance with aspects of the present invention;

FIG. 14E is a TEM image of nanoparticles in accordance with aspects of the present invention;

FIG. 15A is a TEM image of nanoparticles 6 months after synthesis in accordance with aspects of the present invention;

FIG. 15B is a photograph of nanoparticles at different pHs in accordance with aspects of the present invention;

FIG. 15C is a graph depicting degradation of nanoparticles in 10% serum (37° C.) in accordance with aspects of the present invention;

FIG. 15D is a TEM image of cells incubated with nanoparticles in accordance with aspects of the present invention;

FIG. 15E is a graph depicting an ICP-MS analysis of media over macrophages after incubation with nanoparticles in accordance with aspects of the present invention;

FIG. 15F is a TEM image of AuGlu after release from PCPP in accordance with aspects of the present invention;

FIG. 16A is a graph depicting the viability of cells incubated with nanoparticles at different concentrations in accordance with aspects of the present invention;

FIG. 16B is a graph depicting the viability of cells incubated with nanoparticles at different time delays in accordance with aspects of the present invention;

FIG. 17 is a graph depicting the attenuation of GSAN versus other contrast media calculated from CT images in accordance with aspects of the present invention;

FIG. 18A is a TEM image depicting individual silver sulfide nanoparticles (Ag₂S-NP) in accordance with aspects of the present invention;

FIG. 18B is a TEM image of a high payload of Ag₂S-NP in accordance with aspects of the present invention;

FIG. 19 is a depiction of a multimodal nanoprobe in accordance with aspects of the present invention;

FIG. 20A is a TEM image of a multimodal nanoprobes in accordance with aspects of the present invention;

FIG. 20B is a SEM image of a multimodal nanoprobes in accordance with aspects of the present invention;

FIG. 20C is a graph depicting the UV-vis absorption spectrum for the multimodal nanoprobes in accordance with aspects of the present invention;

FIG. 20D is a graph depicting the EDX spectrum of the multimodal nanoprobes in accordance with aspects of the present invention;

FIG. 21A is a collection of dual energy mammography phantom images in accordance with aspects of the present invention;

FIG. 21B is a collection of computed tomography phantom images in accordance with aspects of the present invention;

FIG. 21C is a graph depicting attenuation rate for CT imaging in accordance with aspects of the present invention;

FIG. 22 is an image of MRI phantom imaging showing decay of signal in accordance with aspects of the present invention;

FIG. 23A is a graph depicting fluorescence of multimodal nanoprobes in accordance with aspects of the present invention;

FIG. 23B is an image of fluorescence displayed by multimodal nanoprobes in accordance with aspects of the present invention;

FIG. 24A is a graph depicting silver leaching of Ag₂S-NP from a multimodal nanoprobe as compared to AgNP in accordance with aspects of the present invention; and

FIG. 24B is a graph depicting biocompatibility of a multimodal nanoprobe in accordance with aspects of the present invention.

### DETAILED DESCRIPTION OF THE INVENTION

Aspects of the invention relate to nanoparticles for use as a contrast agent, kits for contrast imaging, as well as methods for dual energy x-ray imaging.

The inventors have recognized that it would be useful to provide contrast agents which are highly effective for DE mammography and other x-ray-based imaging techniques such as conventional mammography, tomosynthesis, fluoroscopy and computed tomography. The inventors have further recognized that it would be useful to provide alloyed nanoparticles as contrast agents for x-ray imaging which are biocompatible as well as excretable via the kidneys. The inventors have also recognized that the inventive contrast agents can optimize DE mammography methods through radiation dose savings, enhanced delineation of the contrast agents, and reduction of the voltage supplied by the high-energy image tube.

As used herein, the term “subject” can be any suitable mammal, including primates, such as monkeys and humans, horses, cows, cats, dogs, rabbits, and rodents such as rats and mice. In one embodiment, the mammal to be imaged in the methods provided herein is a human.

As used herein, the terms “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art.

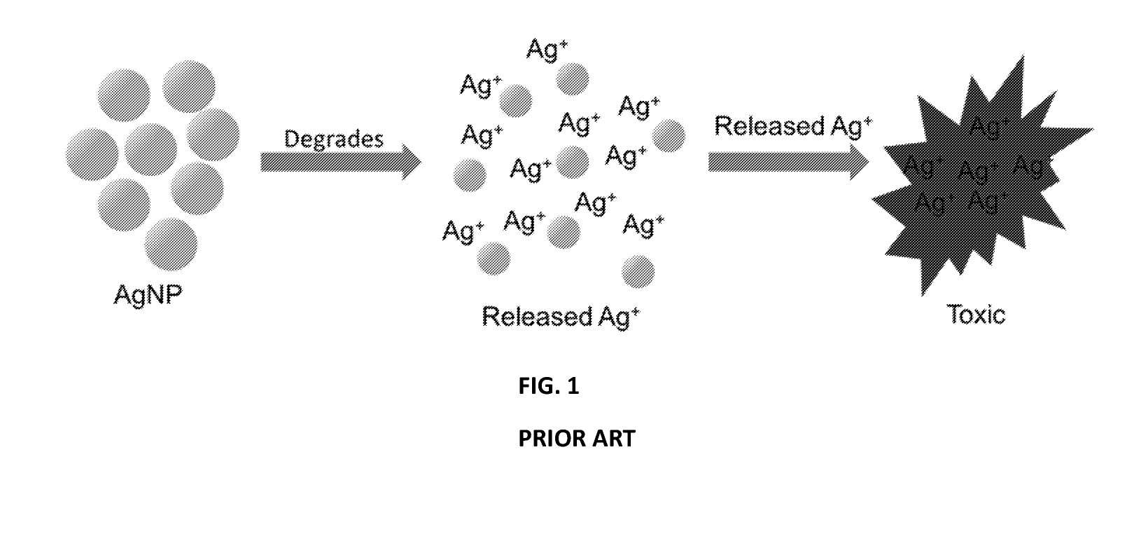

FIG. 1 is a schematic illustration of a prior art process through which a contrast agent including silver nanoparticles degrades once injected into the subject for which x-ray imaging is to be performed. The silver nanoparticles are not stable towards oxidation, which can result in leaching of silver ions. The degradation into silver ions presents safety concerns for the patient, i.e., cytotoxicity and immunoreactivity.

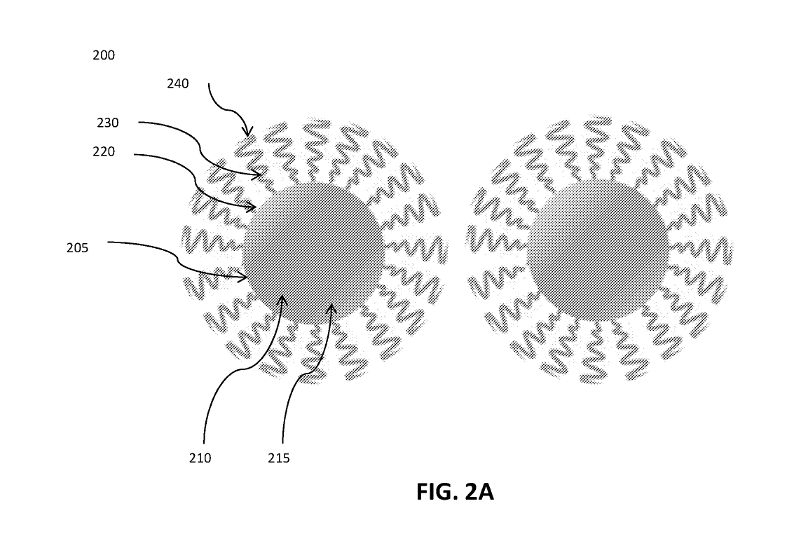

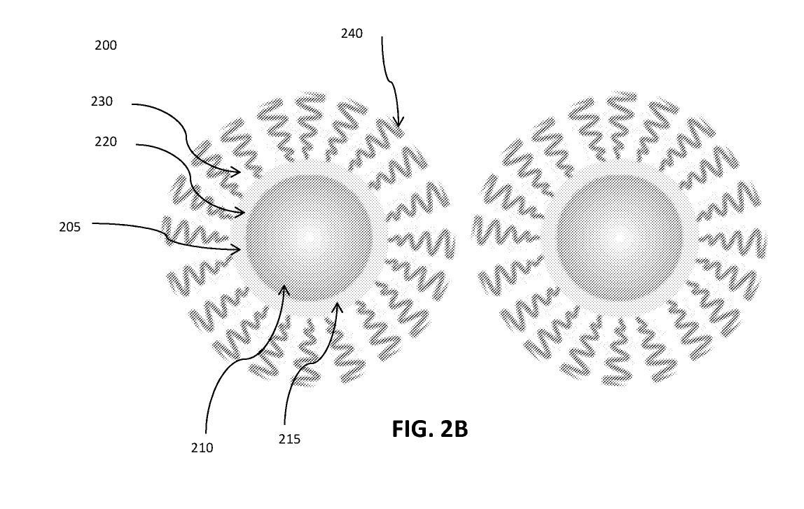

Turning to FIG. 2A, nanoparticles 200 for use as a contrast agent for medical imaging according to the present invention are depicted. Generally, nanoparticles 200 include a core 205 and one or more coating layers. Core 205 includes a contrast element 210 and a stabilizing element 215.

Contrast element 210 preferably has a K-edge value ranging from about 17 to about 49 keV. While this k-edge value range performs well with x-ray imaging, one of ordinary skill in the art will understand, upon reading this disclosure, that other k-edge values may be used without departing from the principles of the present invention. In one embodiment, contrast element 210 is one or more of Zr, Nb, Mo, Tc, Ru, Rh, Pd, Ag, Cd, In, Sn, Sb, Te, I, Cs, Ba, La, Ce, Pr, Nd, Pm, Sm and Eu. Contrast element 210 may exist in core 205 as an element or as a compound. For example, contrast element 210 may exist as elemental silver, or as Ag₂S.

Stabilizing element 215 may be selected to minimize one or both of cytotoxicity and immunoreactivity of the contrast element. Suitable stabilizing elements include B, C, N, O, F, Al, Si, P, S, Cl, Ti, V, Cr, Mn, Fe, Co, Ni, Cu, Zn, Ga, Ge, As, Se, Br, Y, Zr, Nb, Mo, Ru, Rh, Pd, Ag, Cd, In, Sn, Sb, Te, I, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Bi, or others. One of ordinary skill in the art will understand that the identity of stabilizing element 215 will depend, in part, on the chemistry of contrast element 210. As with contrast element 210, stabilizing element 215 may exist in core 205 as an element or as a compound.

In one embodiment, depicted by FIG. 2A, stabilization of the contrast element may be achieved by alloying together the contrast element and stabilizing element. Other contrast element-stabilizing element configurations can result in stabilization and provide, e.g., the desired reduction in cytotoxicity and/or immunoreactivity. Turning to FIG. 2B, core 205 includes a contrast element core 210 and an encapsulating stabilizing element 215. In yet another embodiment, the contrast element and the stabilizing element may react to form a compound, or the stabilizing element may form a compound with a third element.

In another embodiment, stabilizing element 215 may be selected so as to contribute to the contrast generation in addition to the contrast element.

In some embodiments, contrast element 210 forms 1% to 99% by weight of core 205. For example, contrast element 210 may form 90%, 80%, 70%, 60%, 50%, 40%, 30%, 20%, 10%, or any number in between these points, by weight of core 205. Similarly, stabilizing element 215 forms from 1% to 99% by weight of core 205. For example, stabilizing element 215 may form 90%, 80%, 70%, 60%, 50%, 40%, 30%, 20%, 10%, or any number in between these points, by weight of core 205.

In one embodiment, the contrast element is Ag and the stabilizing element is Au. The inventors discovered that silver is an excellent choice as an element upon which to base DE mammography specific contrast agents, because silver's contrast to noise ratio (“CNR”) in DE mammography is close to the maximum, while the dose fraction for silver is approximately evenly weighted between the HE and LE acquisitions, which is necessary to produce images of acceptably low noise and low dose. As described above, the inventors determined that the challenge for translation of silver nanoparticles is that they are not stable towards oxidation, which can result in leaching of silver ions. The inventors surprisingly discovered that inclusion of small quantities of gold in the formulation will raise the oxidation potential of the silver, preventing leaching of silver ions and making the nanoparticles biocompatible. The inventors further discovered that gold nanoparticles have an excellent biocompatibility record and cannot be oxidized in vivo.

It should be noted that, while gold-silver alloyed nanoparticles (“GSAN”), silver sulfide nanoparticles, and Ag2S-NP/IO-NP nanoprobes may be used from time to time to exemplify certain principles of the present invention, the present invention is not so limited. Rather, as described throughout the specification, one of ordinary skill in the art will understand that the present invention includes various other contrast element(s) and stabilizing element(s). The present invention also envisions, and one of ordinary skill in the art will understand based upon this disclosure, other relationships between the contrast element and the stabilizing element, which result in overall stabilization of the nanoparticles beyond alloying, compounding, and the core-shell configuration described above.

Turning back to FIG. 2A, a first coating layer 220 encapsulates core 205. In one embodiment, first coating layer 220 is configured to render nanoparticles 200 soluble in a biological medium such as, e.g., serum. First coating layer 220 may include one or more first coating layer components such as small molecules, peptides, sugars, lipids, phospholipids, proteins, and polymers. One of ordinary skill in the art will understand that a variety of components will render nanoparticles 200 suitably soluble in biological media including, but not limited to, glycerol, polyethylene glycerol, lipoic acid, oleic acid, glutathione, dodecanethiol, polyethylene glycol modified phospholipids, 11-mercapto-undecanoic acid, thioglucose, lecithin, dimyristoyl phosphatidylcholine, albumin, apolipoprotein AI, thio-polyethylene glycol, polyacrylic acid, poly(D,L-lactic-co-glycolic acid), polycaprolactone, poly(vinyl-pyrrolidone), poly(acryl-amide), and poly(glycerol).

In one embodiment, second coating layer 230 encapsulates first coating layer 220. Second coating layer 230 may be configured to delay an in vivo release of core 205 (e.g., the contrast payload). One of ordinary skill in the art will understand that a variety of biodegradable carrier matrices and other excipients may be used to achieve a delayed release of core 220. In certain embodiments, the one or more biodegradable carrier matrices include spermine and polyphosphazene or polyphosphazene derivatives.

In other embodiments, third coating layer 240 may include one or more fluorophores and/or targeting moieties. Fluorophores include, e.g., molecules such as rhodamine, Cy5.5, Alexa dyes, squarines. Targeting moieties include, e.g., small molecules, sugars, aptamers, peptides, proteins and antibodies and others known to those of ordinary skill in the art.

One or more of the coating layers may be omitted while still remaining within the teaching of this disclosure (e.g., first coating layer 220 may be omitted, but second coating layer 230 could remain). Similarly, the ordering of the coating layers described above could be altered without departing from the principles of the present invention (e.g., first coating layer 220 encapsulates second coating layer 230, etc.).

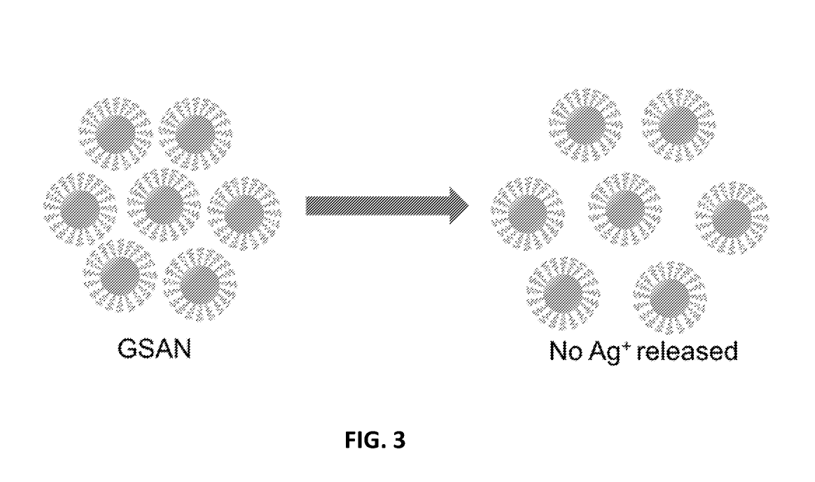

Comparing the prior art process of FIG. 1, employing prior art silver nanoparticles, with FIG. 3, employing the inventive nanoparticles 200, it can be seen that cytotoxicity and immunoreactivity are reduced. In particular, the process of FIG. 3 does not result in degradation causing the release of unstable silver ions.

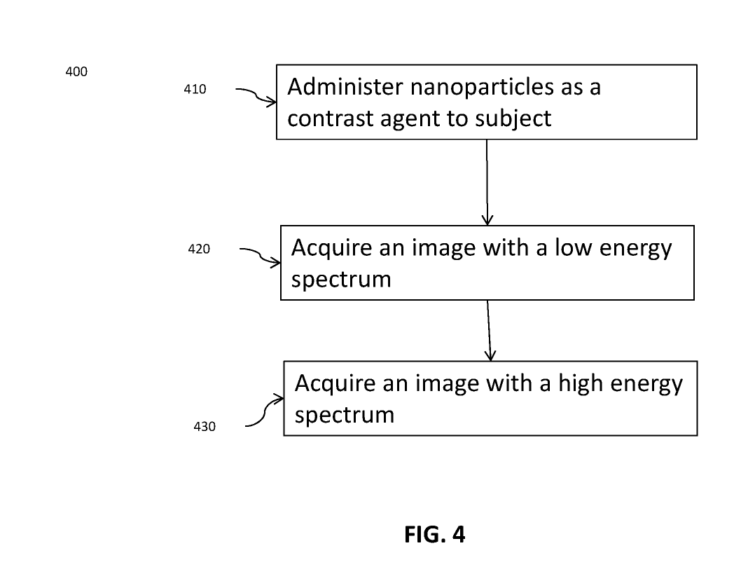

Turning to FIG. 4, a flow diagram depicting selected steps of a process 400 for dual energy x-ray imaging according to aspects of the invention is shown. It should be noted that, with respect to the methods described herein, it will be understood from the description herein that one or more steps may be omitted and/or performed out of the described sequence of the method (including simultaneously) while still achieving the desired result.

In step 410, the inventive nanoparticles are administered to a subject (e.g. nanoparticles 200).

In step 420, an image is acquired using a low energy spectrum. Dual-energy imaging involves acquiring images at two distinct energy spectrums (low and high). Weighting factors are then applied to create an image where the contrast between background tissues has been suppressed. Acquiring an image at a low energy spectrum may include filtering with one or more of a molybdenum filter, a rhodium filter, a silver filter and combinations thereof.

In step 430, an image is acquired using a high energy spectrum. In some embodiments, acquiring an image at a high energy spectrum comprises filtering with one or more of a tin filter, an aluminum filter, a copper filter and combinations thereof.

In another embodiment, a kit for contrast imaging according to the present invention is provided.

The kit includes a dry-powder dosage formulation including a contrast agent comprising a plurality of nanoparticles (such as, e.g., nanoparticles 200). The nanoparticles each have a core (such as, e.g., core 205). The core includes both a stabilizing element (such as, e.g., stabilizing element 215) and a contrast element (such as, e.g., contrast element 210). The stabilizing element (e.g., stabilizing element 215) may be one or more of B, C, N, O, F, Al, Si, P, S, Cl, Ti, V, Cr, Mn, Fe, Co, Ni, Cu, Zn, Ga, Ge, As, Se, Br, Y, Zr, Nb, Mo, Ru, Rh, Pd, Ag, Cd, In, Sn, Sb, Te, I, Ba, La, Ce, Pr, Nd, Pm, Sm, Eu, Gd, Tb, Dy, Ho, Er, Tm, Yb, Lu, Hf, Ta, W, Re, Os, Ir, Pt, Au and Bi. The contrast element (e.g., contrast element 210) preferably has a K-edge value ranging from about 17 to about 49 keV. A first coating layer (such as, e.g., first coating layer 220) encapsulates the core and is configured to render the nanoparticles soluble in a biological medium.

The kit also includes a pharmaceutically acceptable carrier solution suitable for injection for reconstituting the dosage formulation.

Pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In some embodiments, parenteral vehicles (for subcutaneous, intravenous, intra-arterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In some embodiments, the inventive nanoparticles are delivered in a vesicle, e.g., a liposome.

In other embodiments, carriers or diluents used in methods of the present invention include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

### Examples

The following examples are included to demonstrate the overall nature of the present invention.

### GSAN Synthesis

100 mL of deionized water and 50 μL of 0.01 M sodium citrate were placed in aqua regia cleaned glassware. Varying mole fractions of 0.01 M HAuCl₄ and 0.01 M AgNO₃ were added to each solution for a total metal salt concentration of 5.0×10⁻⁶ M. For example, the 50/50 Au/Ag reduction solution consisted of 100 mL deionized water, 50 μL 0.01 M sodium citrate, 25 μL 0.01 M HAuCl₄, and 25 μL 0.01 M AgNO₃. Next, 50 μL of freshly prepared 0.01 M NaBH₄ was added with vigorous stirring. The solution was allowed to stir for an additional 30 s. A color change occurred almost immediately (the color varied from yellow to red depending on the ratio of gold to silver), which correlated with shifting UV/visible absorbance peaks. This procedure was scaled up, the nanoparticles were capped with glutathione. Purification was performed using molecular weight cut-off tubes and the solutions were concentrated to ˜10 mg/ml in PBS.

### PCPP-NP Synthesis with GSAN

PCPP will be dissolved in an aqueous solvent at a concentration of 0.5% and the pH adjusted to 7.4. Stock solutions of spermine will be made in the same solvent (pH 7.4) at a concentration of 2 mg/ml. Spermine is an endogenous substance and has an LD50 via IP injection into mice of 870 mg/kg. The amount of spermine in a dose would be at least 100 times less than that, indicating the safety of this reagent. Spermine has four amines, which can be protonated at physiological pH and crosslink the polycarboxylate PCPP to form nanoparticles. 1 ml of PCPP solution, diluted to the desired concentration, will be mixed with the loading material. For example, 0.5 ml of 80%-Ag, glutathione coated GSAN (10 mg Ag/ml, deionized water) was added to 1 ml of 0.2% PCPP to create the nanoparticles. Co-injection of the PCPP-GSAN and spermine solutions into a herringbone patterned microfluidic chip allows the synthesis of PCPP-NP that are homogeneous in size. The resulting suspension will be immediately added to 100 ml of 8.8% CaCl₂ solution. After stirring for 30 minutes, the nanoparticles are isolated by centrifugation and washed with deionized water, which will remove any non-entrapped GSAN.

### GSAN Synthesis and Fabrication

Turning to FIG. 5, a schematic illustration of the preparation of sub-5 nm GSAN is depicted according to aspects of the present invention. The gold-silver nanoparticles, due to their size, should accumulate in tumors, allowing them to be easily detected.

It has been shown that nanoparticles larger than 5 nm will be retained within the body, potentially for years, whereas nanoparticles smaller than 5 nm can be swiftly excreted via the kidneys and the urine. However, some of the critical strengths of nanoparticles, i.e. long circulation times and accumulation in tumors, arise due to their large size (>5 nm) preventing swift urinary excretion of nanoparticles. The long-term retention of contrast agents would be a concern that would likely prevent their eventual clinical application. The most optimal formulation of long-circulating GSAN, therefore, would be larger than 5 nm, but would slowly break down into sub-5 nm components that could be excreted via the urinary system. This approach would result in low concentrations of GSAN reaching the kidneys over an extended time, which would minimize any potential nephrotoxicity. Therefore such nanoparticles might be more biocompatible for patients with renal disease, due to the low kidney concentration at any time. Some of the nanoparticles will be taken up in the liver, but data indicates that the polymer will be degraded in the liver and the metal cores either excreted via the kidney or the feces.

As shown in FIG. 5, the polyphosphazene used in this preparation, poly-di(carboxylatophenoxy)phosphazene, degrades into harmless byproducts, i.e. phosphate, ammonia (natural biological substances) and 4-hydroxybenzoic acid (a substance in various foodstuffs such as wine, with an LD50 of 2200 mg/kg in mice). These particles would maintain their size and structure in the short-term (˜24 hrs) after injection, allowing their diagnostic potential to be fulfilled. After this period, the polymer will degrade and release the small GSAN, which will be excreted.

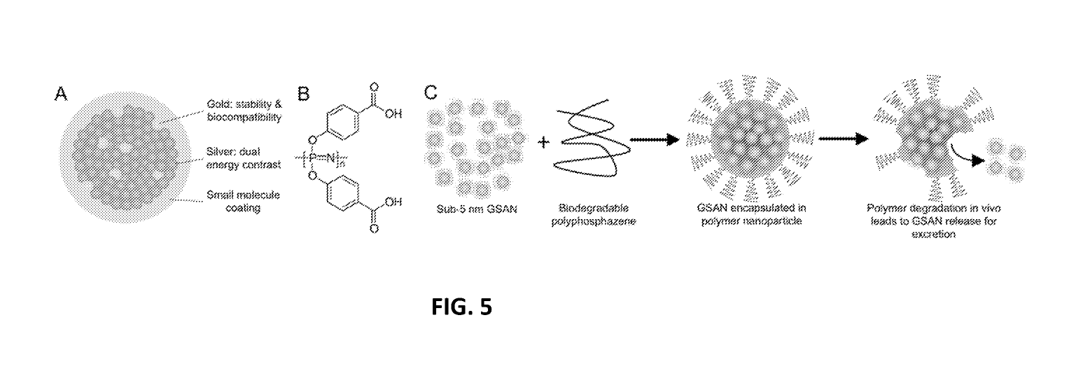

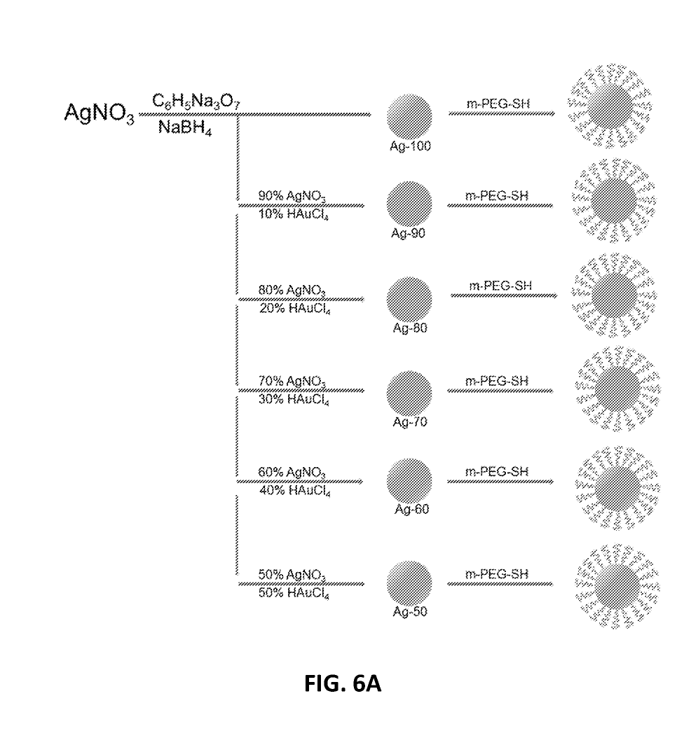

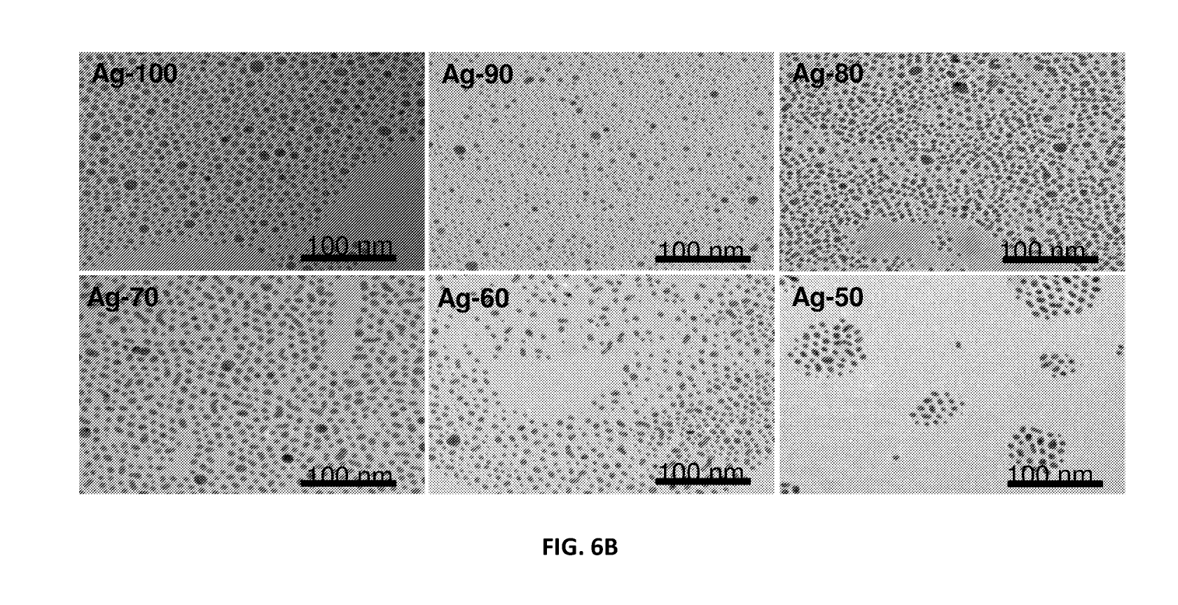

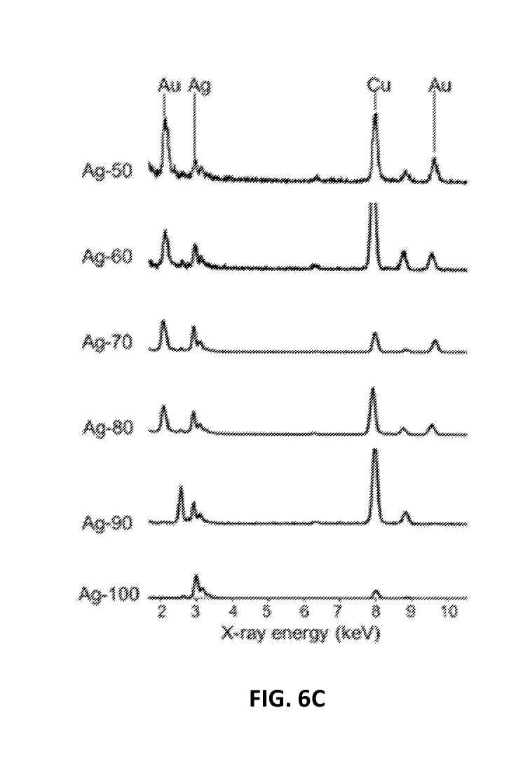

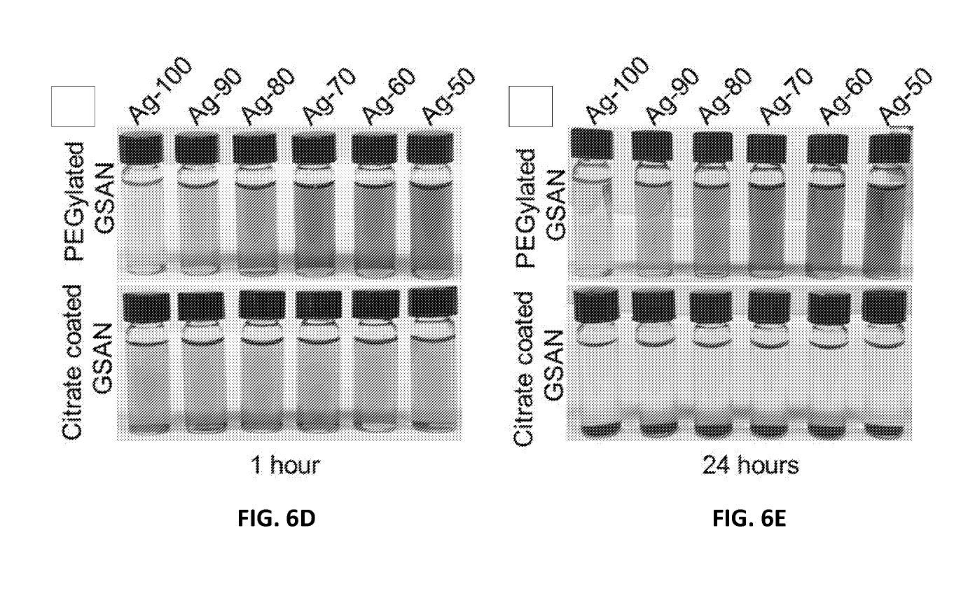

As shown in FIG. 6A, GSAN may be synthesized via reduction of silver nitrate and chloroauric acid by addition of sodium borohydride in the presence of citrate. Six different formulations (termed Ag-100, Ag-90, Ag-80, Ag-70, Ag-60 and Ag-50) were synthesized by adjusting the molar ratio of gold and silver salts. Ag-100 refers to the formulation where 100% silver was used in the synthesis, whereas Ag-90 refers to the formulation where 90% silver and 10% gold used in the synthesis and so on. Table 1 (below) describes the different molar ratios used in the different formulations, along with characteristics of each formulation. The core size of all the GSAN formulations was found to be around ˜5 nm. The hydrodynamic diameters and zeta potentials of all the GSAN formulations were found to be around 16 nm and −20 mV, respectively (Table 1). The core and hydrodynamic diameters of all the GSAN formulations were similar, as the synthesis methodology was similar for all the formulations, with only the ratio of silver and gold salts varying.

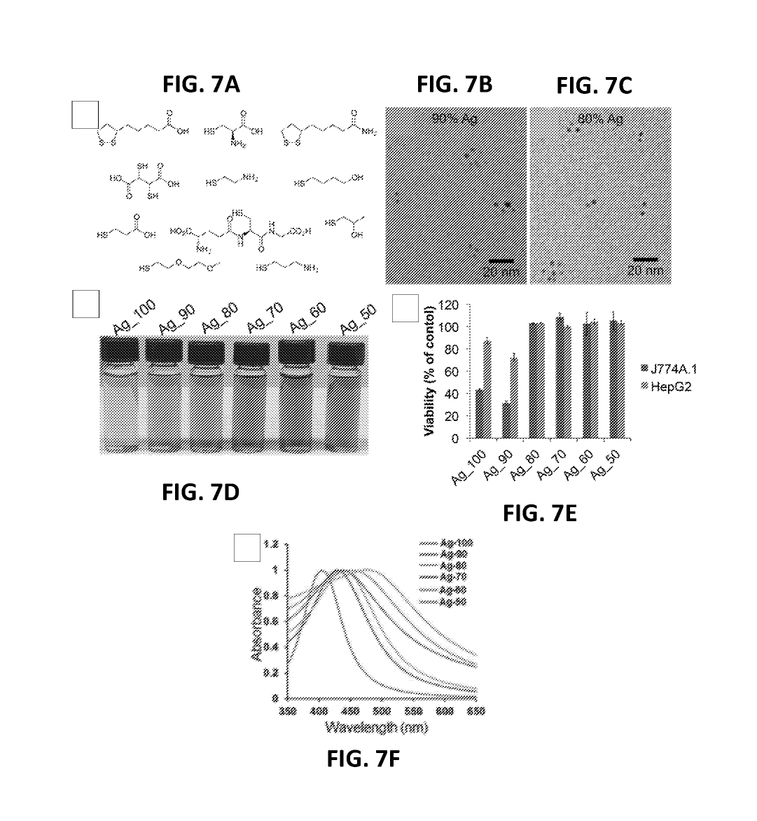

Surface modification of these nanocrystals may be used to provide solubility and stability in biological media. Incubation with the relevant ligand is followed by purification via centrifugation. If certain ligands cannot easily be directly coated onto nanoparticles synthesized via this method, two-step substitution methods may be used where thioctic acid or Tween 20 is used as an intermediate coating prior to addition of the final coating. FIG. 7A depicts numerous potential GSAN variations by using single ligands, 1:1, 1:3 and 3:1 mixtures of the ligands (9 single ligand coatings, and 36 each for 1:1, 1:3 and 3:1, giving a total of 9+36+36+36=117). The coating ligands will contain a thiol group at one end for binding to the gold/silver surface and other functionalities, such as carboxylic acid groups, amines, amides and alcohols at the other end. These ligands were chosen in view of the expected excellent water solubility as well as hydrogen bonding groups that will facilitate inclusion into PCPP NP. The GSAN may be tested for their stability in biological media by incubating with 10% serum for 24 hours and measuring their size with DLS. Either loss of coating or opsonization should result in aggregation and hence size increases.

The particles of the six different GSAN formulations were capped with thiol-polyethylene glycol (PEG) with a methoxy end group (FIG. 6A) to make them water soluble, stable in physiological buffer conditions and to enhance the in vivo blood circulation half-life. PEGylated GSAN formulations were found to be stable in PBS, as PEG-coated GSAN remained well suspended in PBS after both 1 hour and 24 hours (FIGS. 6D and 6E). In contrast, citrate-coated GSAN formulations were not found to be stable in PBS (FIGS. 6D and 6E). PEG provides gold nano-particles with robust stability in biological media. These sub-5 nm GSAN synthesized with a range of silver to gold percentages and are shown in FIGS. 7B-7D. The color of the formulations changes from yellow to reddish-brown as the amount of gold increases (FIG. 7D). This corresponds to a shift in the absorption maximum from 405 nm for Ag-100 to 485 nm for Ag-50 (FIG. 7F). The coating material for the all the formulations was the same (i.e., m-PEG), therefore there were no substantial differences in zeta potential values among the formulations (Table 1). Table 2 (below) describes the relevant synthesis conditions for the formulations. FIG. 6B depicts TEM images of GSAN produced according to these synthesis techniques.

ICP-OES was performed on all the GSAN formulations (Table 1). The results show that significant amounts of gold and silver were found in all the GSAN formulations except Ag-100. Energy dispersive X-ray spectroscopy (FIG. 6C) confirmed the presence of silver and gold in the GSAN core (copper peaks are due to the grid material). The yield of silver was found to be around 60-70% (Table 1).

TABLE 1
Synthesis and characterization data for GSAN formulations

| | 0.1M | 0.1M | Hydrodynamic | Core diameter | Zeta potential | Molar ratio | Molar | % yield |
|---|---|---|---|---|---|---|---|---|
| Formulation | AgNO₃ (μl) | HAuCl₄•3H₂O (μl) | diameter (nm) | (nm) | (mV) | (Ag/Au) | (%) Ag | (Ag) |
| Ag-100 | 1250 | 0 | 13 ± 2 | 7.6 ± 2.8 | −18.1 ± 1.9 | — | — | 63 |
| Ag-90 | 1125 | 125 | 16 ± 1 | 3.6 ± 1.4 | −19.3 ± 2.8 | 5.97 | 85.6 | 68 |
| Ag-80 | 1000 | 250 | 16 ± 5 | 4.7 ± 2.3 | −21.5 ± 2.7 | 2.41 | 70.6 | 69 |
| Ag-70 | 875 | 375 | 17 ± 2 | 6.4 ± 1.7 | −19.6 ± 1.3 | 1.74 | 63.5 | 70 |
| Ag-60 | 750 | 500 | 14 ± 3 | 4.6 ± 1.4 | −20.0 ± 1.8 | 1.08 | 51.9 | 70 |
| Ag-50 | 625 | 625 | 16 ± 4 | 5.7 ± 1.5 | −20.2 ± 1.1 | 0.66 | 39.7 | 58 |

TABLE 2
SYNTHESIS CONDITIONS OF GSAN

| | | | Sodium | Sodium |
|---|---|---|---|---|
| | Silver nitrate | Gold Chloride | citrate | borohydride |
| | 0.1M (μL) | 0.1M(μL) | 0.1M (μL) | 0.1M (μL) |
| Ag-100 | 1250 | 0 | 1250 | 5000 |
| Ag-90 | 1125 | 125 | 1250 | 5000 |
| Ag-80 | 1000 | 250 | 1250 | 5000 |
| Ag-70 | 875 | 375 | 1250 | 5000 |
| Ag-60 | 750 | 500 | 1250 | 5000 |
| Ag-50 | 625 | 625 | 1250 | 5000 |

Alternatively, GSAN can be synthesized by reduction of the salts with sodium borohydride in toluene and in the presence of a molar excess of thiols, producing cores of 1-3 nm in diameter. A relatively short chain thiol, such as heptanethiol, may be used, which will allows the coating to be substituted for a variety of molecules. After ligand substitution, the resulting nanoparticles will be isolated via centrifugation, washed and resuspended in deionized water. In an additional alternative approach, silver cores can be formed and a gold shell grown on them via a growth solution.

### Assessment of Biocompatibility of GSAN

Unlike gold nanoparticles, silver nanoparticles are not inert in nature and can release silver ions, which can be a safety concern (FIG. 1). It was surprisingly found that making a gold-silver alloy nanoparticle increases the reduction potential of silver atoms in the nanoparticle, minimizing silver ion release and improving biocompatibility (FIG. 3). Silver ion leaching from all GSAN formulations was tested in two different dissolution media: DI water and simulated lysosomal fluid (pH 5) at 37° C. Simulated lysosomal fluid was used because it has frequently been observed that nano-particles are internalized into endosomes or lysosomes. The lysosomal pH is slightly acidic, at around pH 5. Since silver nanoparticles release more silver ions in acidic pH than neutral pH (7.4), probing lower pH is particularly important. Therefore to probe pH dependence silver ion release assays were performed in these two different dispersion media (i.e. neutral and slightly acidic pH). Cytotoxicity assays with two different cell types were also performed to probe the correlation of silver ion release data with cytotoxicity. For example, the biocompatibility of 3 nm glutathione coated GSAN is depicted in FIG. 7E, where it can be seen that the inclusion of >10% Au in this preliminary formulation eliminates toxicity in J774A.1 mouse macrophage cells and HepG2 human liver cells.

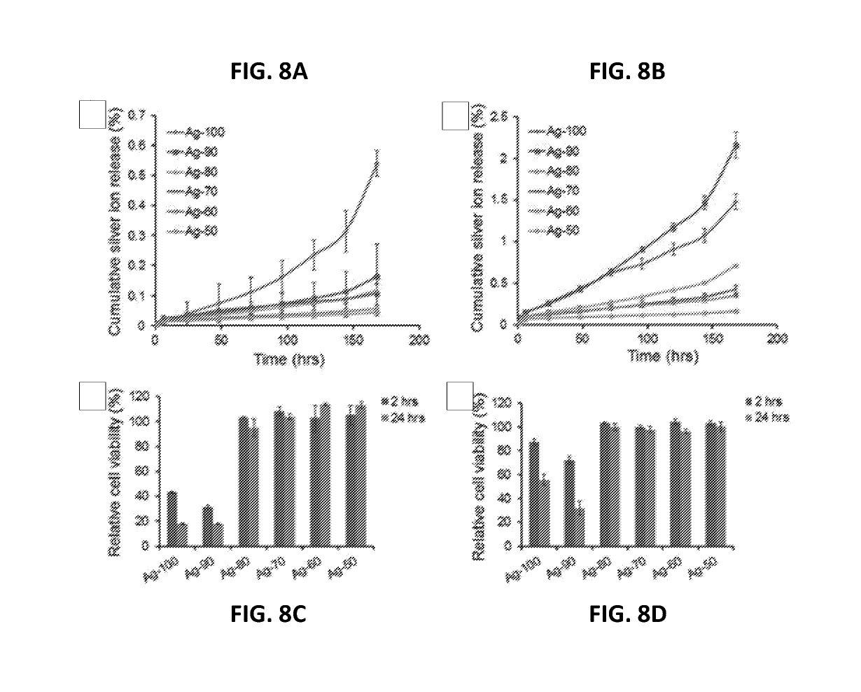

The release of silver ions from GSAN compared with silver nanoparticles is shown in FIGS. 8A and 8B. FIG. 8C and FIG. 8D depict, respectively, cell viability study results for GSAN with respect to J774A.1 and HepG2 cells. In the case of DI water, more silver ions were released from silver nanoparticles (Ag 100) than GSAN formulations, with a trend that less silver is released the more gold is included in the formulation (FIG. 8A). While about 0.6% of the silver was released from Ag-100 over 7 days, less than 0.1% release was seen from GSAN with 20% or more gold content. In simulated lysosomal fluid, Ag-100 and Ag-90 formulations release substantially more silver ions than the other GSAN formulations (FIG. 8B). Surprisingly, Ag-90 released more silver ions than Ag-100, in contradiction to the results found in DI water. However, this data matches well with results from the cell viability assay (see FIGS. 8C and 8D). Inclusion of 20% gold or more resulted in substantially decreased silver ion release, with 0.6% release or less, whereas 1.5% silver was released from Ag-100. As can be seen, more silver was released under acidic conditions than neutral conditions, which corresponds with the results found by others.

The impact of the GSAN formulations on the viability of J774A.1 and HepG2 cells was explored. All the GSAN formulations were found to be biocompatible with both cell lines except Ag-100 and Ag-90 (FIGS. 8C and 8D). Substantial reductions of J774A.1 and HepG2 cell viability were observed when incubated with Ag-100 and Ag-90 formulations. This result is likely due to the greater amount of silver ions leached from these nanoparticles compared to other GSAN formulations. Ag-100 is a pure silver nanoparticle and the observed decreases in cell viability are in line with the results found by others. Surprisingly, when incubated with Ag-90, the cell viability was even lower than for Ag-100. This result is in agreement, however, with the results found for silver ion release.

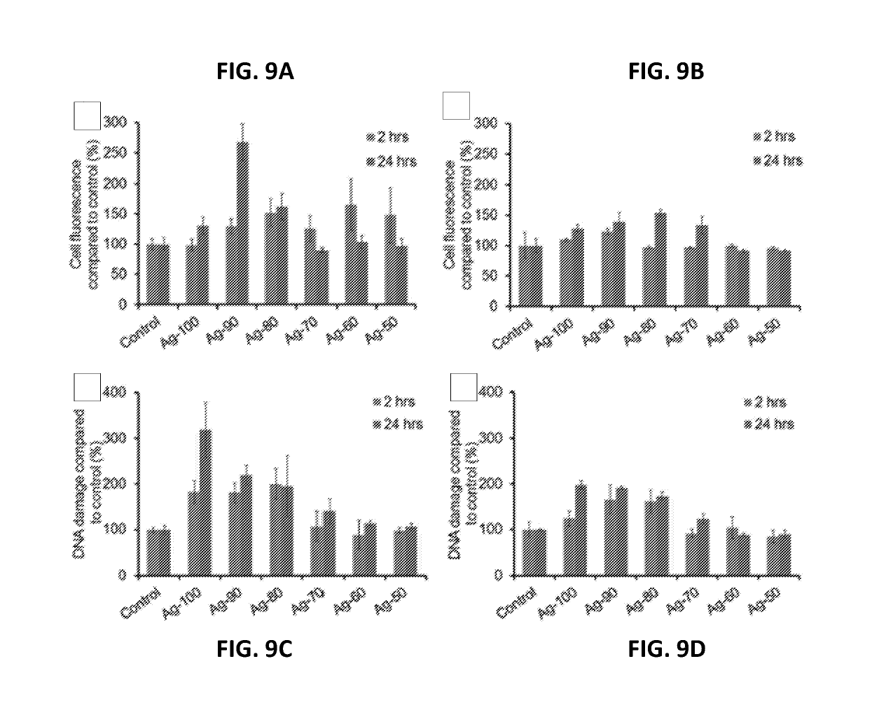

More in-depth toxicological assessments of GSAN were performed via measurement of reactive oxygen species (ROS) generation and DNA damage studies in J774A.1 and HepG2 cells. The results of these experiments are presented in FIG. 9. Notably increased ROS generation was found in J774A.1 cells when incubated with Ag-100, Ag-90 and Ag-80 formulations, but not when incubated with Ag-70, Ag-60 and Ag-50. ROS generation was lower in HepG2 cells, but followed the same trend as for J774A.1, i.e., that Ag-100, Ag-90 and Ag-80 resulted in increased ROS generation, whereas Ag-70, Ag-60 and Ag-50 did not. DNA damage was found in 3774A.1 and HepG2 cells with the Ag-100, Ag-90 and Ag-80 formulations. However, only minor DNA damage was observed with both cell lines after 24 hours incubation with Ag-70. No DNA damage was found after incubations with the Ag-60 and Ag-50 formulations in either cell type.

Cell viability, ROS and DNA damage results indicate that inclusion of 30% or more gold in the formulation renders the GSAN biocompatible (FIGS. 8 and 9). However, suitable biocompatibility can likely be achieved with 80% silver GSAN, and perhaps with even higher silver content. Improved synthesis methods will likely result in zero toxicity at lower Au inclusion levels. Lead formulations will not increase in size more than 20% upon incubation with serum. These formulations should not lead to significant changes in cell viability, cytokine release, cell spreading, oxidative stress, unfolded protein response or red blood cell lysis compared with control, as supported by our preliminary data. The viscosity should be less than 25 cps, so as to be injectable under relevant flow rates (4 ml/s) and the osmolality should generally not be greater than that of 120% of blood (290 mOsm/kg).

### De Mammography and CT Scanning Properties of GSAN

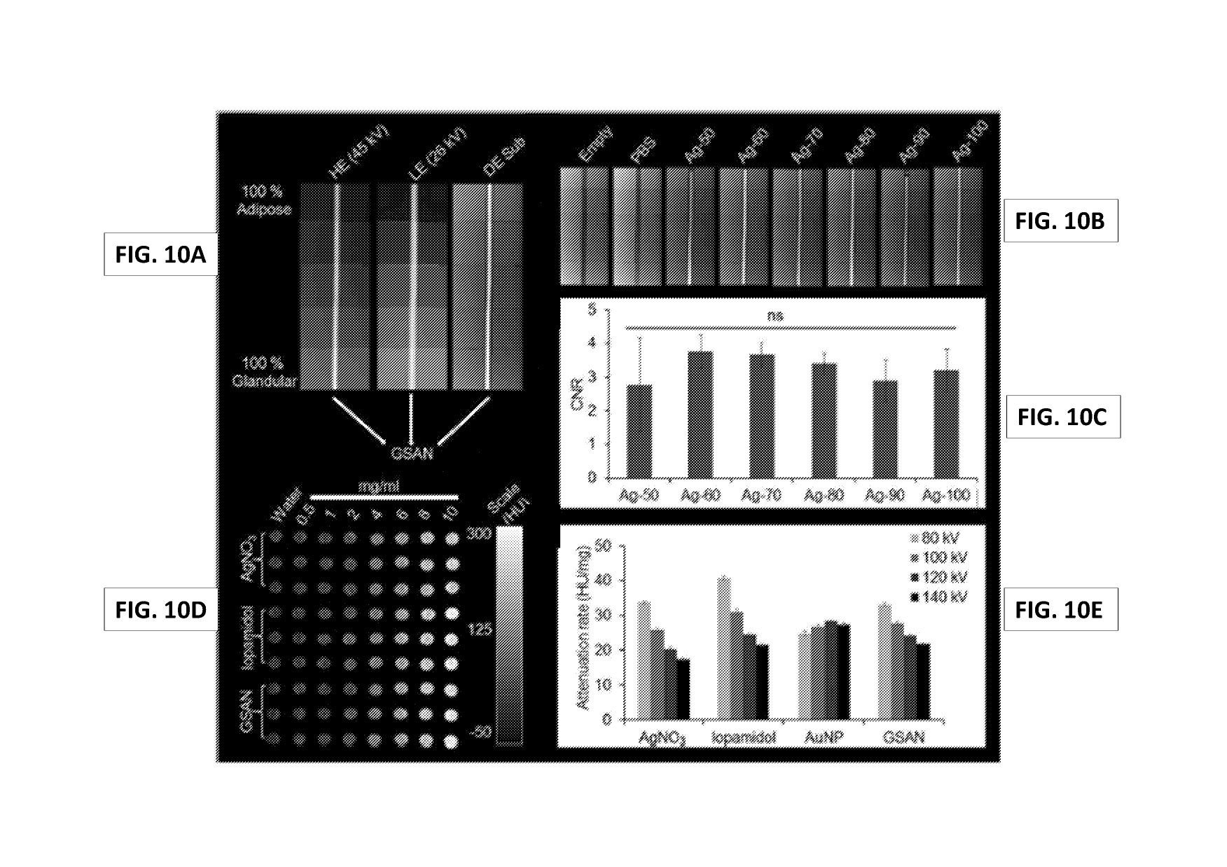

Phantom imaging to ascertain that the addition of gold would not affect the DE mammography contrast properties of silver was also performed. The generation of contrast in DE mammography depends on an element having a k-edge in the high energy window, e.g. from roughly 20-40 keV. Since the k-edge of gold is at 80.7 keV, gold was not predicted to generate significant DE mammography contrast. The DE mammography contrast properties of GSAN (at 16 mg Ag per ml) were investigated using a step phantom composed of materials that provide density ranging from that of 100% adipose to 100% glandular tissue. DE mammography images of this phantom acquired using a Hologic Selenia Dimensions DE mammography system are presented in FIGS. 10A and 10B. Images were acquired at low energy (LE) and high energy (HE). A weighted logarithmic subtraction was performed to obtain dual-energy (DE) images (FIG. 10A); it can be seen that the variation in the background signal has been greatly reduced and the contrast from the GSAN is enhanced. Strong DE mammography contrast was observed from all the GSAN formulations and the silver nanoparticles (FIGS. 10B and 10C). The contrast-to-noise ratios (CNR) for all formulations were calculated from the DE mammography phantom images and are presented in FIG. 10C. The results indicate that there is no significant difference in the CNR produced among all these formulations, as the concentration of silver was held constant for all the formulations (FIG. 10C). Therefore, DE mammography phantom imaging results indicated that inclusion of gold in GSAN did not affect the DE mammography contrast properties created by silver.

The CT contrast properties of GSAN were evaluated using a clinical CT scanner. An FDA-approved iodine-based contrast agent (iopamidol), gold nanoparticles and silver nitrate were also scanned as controls. Selected CT phantom images are presented in FIG. 10D, while the CT attenuation rate of the different agents is presented in FIG. 10E (the GSAN formulation is Ag-80). The CT phantom results show that silver attenuates X-rays comparably to iopamidol, with the greatest attenuation at 80 kV, followed by a steady decline in attenuation as the X-ray tube voltage increases to 140 kV. Since the k-edge of silver is at 26 kV, silver should have stronger attenuation of lower energy X-rays than higher energy X-rays. These results indicate that silver has potential as a CT contrast agent in addition to a contrast agent for DE mammography. Gold has a k-edge at 80.7 kV, therefore it has stronger attenuation of high energy X-rays than low energy X-rays, hence the increase in attenuation from 80 to 120 kV. GSAN (Ag-90 to Ag-50 formulation) attenuates high energy X-rays with an absorption that is intermediate to those of gold and silver as it contains both of these elements. As can be seen, GSAN create CT contrast that is comparable with iodinated agents and gold nanoparticles and therefore demonstrates usefulness as a CT contrast agent.

### In Vivo De Mammography with GSAN

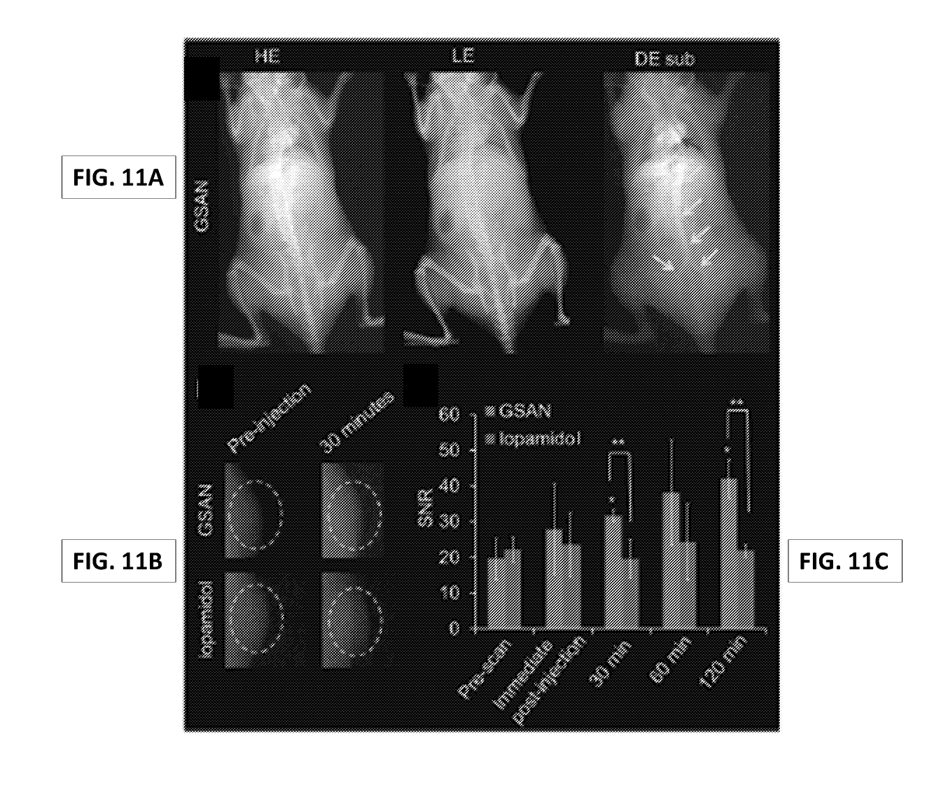

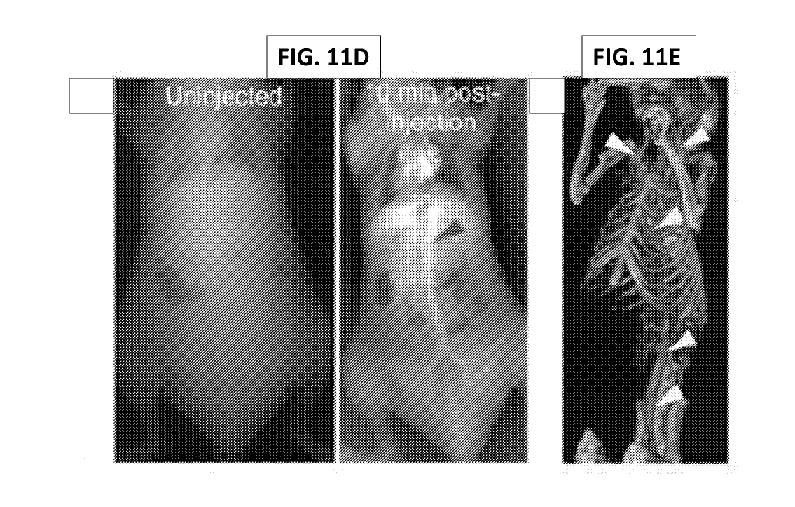

In vivo DEM and CT contrast properties and tumor accumulation efficiency of a GSAN formulation (Ag-60) were investigated. The Ag-60 formulation was selected due to its low silver ion release and lack of adverse effects on cells in vitro. When GSAN were injected intravenously into mice without tumors, strong DE mammography contrast was observed in the heart and major blood vessels at 5 minutes post-injection (FIG. 11A). These results indicated that detection of GSAN with DE mammography imaging was practical in vivo.

Other example image data was derived from mice (without tumors) that were imaged after injection with 80% Ag GSAN (FIG. 11D). As can be seen, the presence of the nanoparticles in bloodstream is easily detected. In addition, the same mice were imaged with CT and found GSAN to produce strong contrast (FIG. 11E). The mice may be anesthetized with isoflurane, catheterized via the tail vein and placed in the scanner. The mice may be prescanned using the afore-mentioned Hologic Selenium Dimensions DE prototype. The ten most biocompatible formulations identified in the above experiments (n=6/group) will be injected via the tail vein at a dose of 100 mg Ag/kg and imaged at 5 min, 30 min, 1 hr and 2 hr. This dose should be effective as nanoparticles frequently accumulate in tumors at >5% ID/g tissue, i.e. 5 mg Ag/g, which should be easily detected in DE mammography. Iopamidol, a clinically approved iodinated small molecule contrast agent, saline and GSAN injections in mice without tumors will be used as controls. The HE images may be registered to the LE images using using ANTS (Advanced Normalization Tools). Mutual information optimization first corrects large-scale motions. Normalized cross-correlation optimization then iteratively corrects fine-scale misalignment. DE images may be calculated as the weighted logarithmic subtraction between the LE and HE images. The signal intensity in the tumors is calculated in each image (LE, HE and DE) via Matlab based image processing tools and the SNR determined. The SNR in the different tumor types for the different nanoparticles and iodine may be compared. Statistical analysis may be performed using SPSS 14.0. A one way Analysis of Variance (ANOVA) with Bonferroni's multiple comparison test may be performed to determine significance differences between the experimental groups as compared to the control group. P-values<0.05 will be considered significant.

Next, in vivo imaging with a mouse model of breast cancer was performed. DE mammography images of representative tumor-bearing mice injected with either GSAN or iopamidol are presented in FIG. 11B. The DE mammography images of the mouse injected with GSAN show that there is an increased DE mammography signal in the tumor compared to the pre-injection scan. On the other hand, for mice injected with iopamidol, there was no substantial difference in the DE mammography signal observed in the tumors between pre- and post-injection scans at this time point (FIG. 11C). The signal-to-noise ratios (SNR) in the tumors indicate that there was a substantial increase in DE mammography signal in the mice injected with GSAN at all post-injection time points (statistically significant at 30 and 120 min) compared to the SNR in the pre-injection scans (FIG. 11C). However, for mice injected with iopamidol, no significant difference in the DE mammography signal was observed in the tumors at any time point. Without being bound to any theory, this is likely because iodine contrast agents have very low circulation half-lives and are quickly cleared from the body via renal excretion. While iodine contrast agents have been used for DE mammography in patients, the clearance is slower in humans than in mice. For example, there was a delay of 1-2 minutes between the injection and first imaging time point, which may be sufficiently long enough for the agent to clear from the tumors in these mice. In contrast, the GSAN are designed to have long circulation half-lives, with enhanced permeability and retention (EPR) effect that should lead to accumulation in the tumor and increase in the DE mammography signal over time.

### In Vivo Imaging of GSAN with CT Scanning

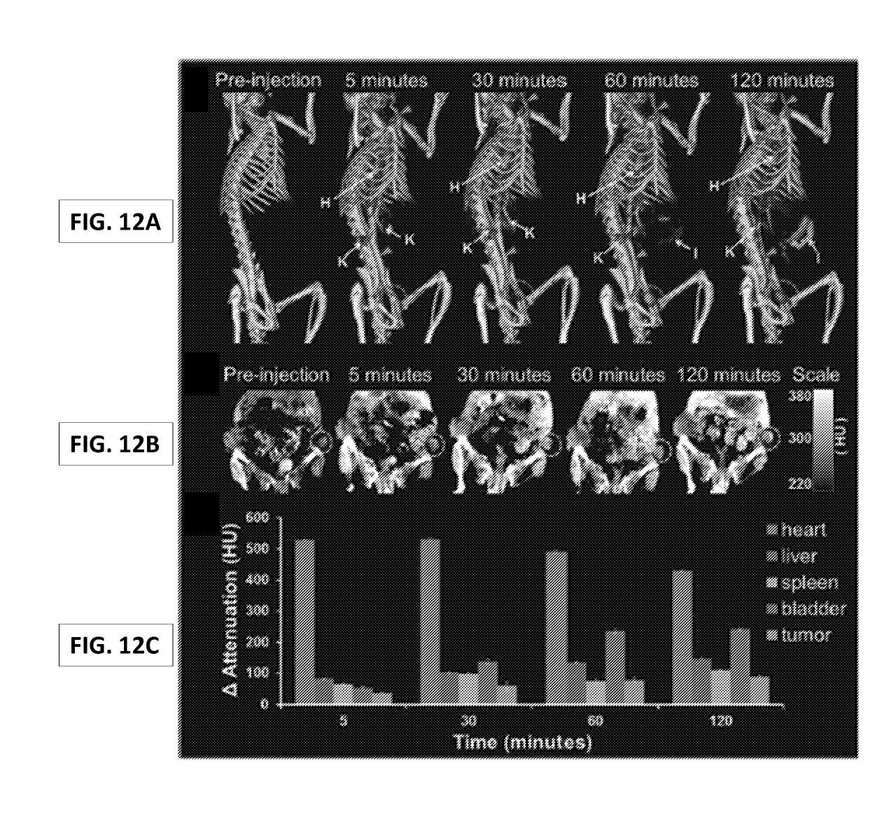

In addition to in vivo DEM imaging experiments, the in vivo CT contrast properties of GSAN were investigated using mice with and without breast tumors. CT images of a mouse without tumors injected with GSAN are shown in FIG. 12A. Contrast was observed in the blood vessels over the imaging time points demonstrating that GSAN produce strong vascular contrast and have a long circulation half-life, as contrast was observed in the blood vessels over the imaging time points.

Contrast was also observed in the bladders and intestines of the mice (FIG. 12A), which suggested that there is some excretion of the GSAN via urine and feces. In addition, CT imaging was done with a breast tumor mouse model and GSAN injections.

The results indicated that GSAN can be used as CT contrast agents for specific detection of breast cancers. In CT images of tumor-bearing mice, significantly higher contrast (p<0.05) was observed in the tumors at all the post-injection time points compared to pre-injection, and the intensity gradually increased over time (FIGS. 12B and 12C). This result is in agreement with the DE mammography imaging. Using this contrast agent (GSAN), CT is suitable for breast cancer screening. In addition, GSAN may be applicable to detect other tumor types such as lung cancer.

The contrast in different organs was quantified over the various time points imaged. The contrast in the heart (i.e., the blood) was highest of all the organs (FIG. 12C), and gradually declined over the two hour imaging time period. Some accumulation in the liver and spleen was observed over time. The contrast observed in the bladder steadily increased over the imaging time period. The detection of contrast in the bladder was surprising, since the hydrodynamic size of the nanoparticles was 14 nm, larger than the expected renal filtration cut-off for nanoparticle excretion.

### Biodistribution and Excretion of Ag-60 GSAN Formulation

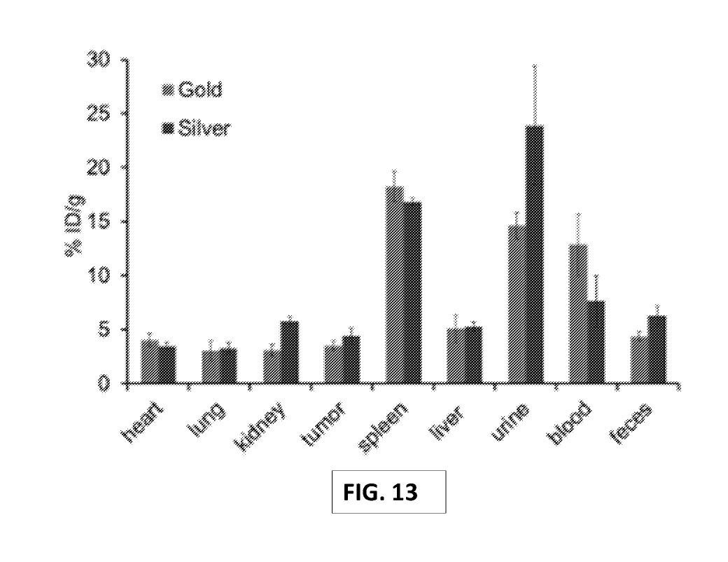

Excretion and biodistribution via ICP-OES of the Ag-60 GSAN formulation was investigated at 2 hours post-injection (FIG. 13) (this time point was selected to match the last in vivo imaging time point in order to confirm the excretion via the urine and feces observed via imaging). Higher amounts of gold and silver were found in the blood and spleen than in other tissues. More than 10% of the injected dose (ID) was still present in the blood, which suggested that these nanoparticles have a reasonably long circulation half-life. Around 5% of the ID was found to be in the tumor, heart, lungs, and liver. Significant amounts of silver and gold were found in the urine and feces, which supports the CT imaging data, and indicates that GSAN are excreted from the body via urine and feces.

### GSAN Encapsulation in Polymer Nanoparticles

GSAN were analyzed for their ability to be encapsulated in biodegradable polyphosphazene matrices. Poly-di(carboxylatophenoxy)phosphazene (PCPP) was used. This polyphosphazene has been shown to degrade over time in aqueous media, via hydrolysis of the polymer backbone. This hydrophilic polymer nanoparticle represents a novel platform for the development of contrast agents. A general method for the synthesis of PCPP-NP is as follows.

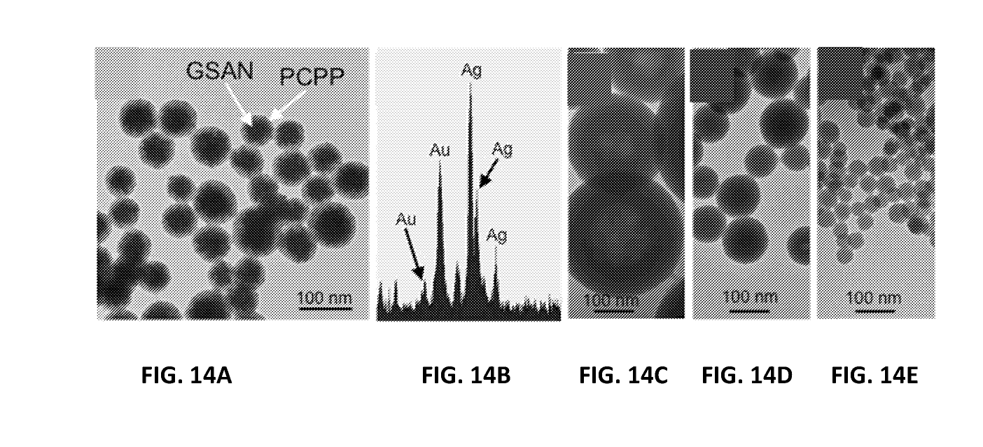

PCPP-NP may be coated with polyethylene glycol (PEG) to provide long circulation half-lives, avoid uptake by the reticuloendothelial system and increase tumor accumulation. Small amounts of polylysine (PLL)-PEG block co-polymers may be included in the synthesis (both blocks 5000 MW). This has the advantage of controlling the nanoparticle size between 30-500 nm by adjusting the PLL-PEG amount used (0.2 to 0.01 mg), as shown in FIGS. 14C-14E. The poly-cation PLL likely binds to the PCPP polycarboxylate, limiting nanoparticle growth. Other possibilities to include PEG are to chemically modify the polymer, chemically modify the spermine or to add PEG molecules that terminate in acids or amines. Size may be controlled via the concentration of the polymer and by varying the amount of PEG included in the synthetic process. By these means nanoparticles of approximately 100 nm in diameter may be produced. The resulting nanoparticles may be sterilized via syringe filtration (0.2 micron), autoclaving the samples or use of gamma irradiation. The GSAN loaded polyphosphazene nanospheres may be characterized with SEM, TEM (FIG. 14A), EDS (FIG. 14B), ICP-MS, zeta potential and DLS (Zetasizer Nano ZS90). Nanocrystal distribution may be examined with electron tomography using, e.g., a FEI Tecnai T12. The gold/silver concentration may be determined via ICP-MS. Quality control on batches may be done with TEM and DLS as above.

### GSAN Loaded PCPP-NP Degradation

PCPP-NP were tested for their stability in biological media by incubating with 10% serum for 1 hour and measuring their size with DLS—an increase in size will indicate a lack of stability. 1 hour should be sufficient time for aggregation to occur, but short enough to avoid significant degradation, which would result in a decrease in size and confound the experiment.

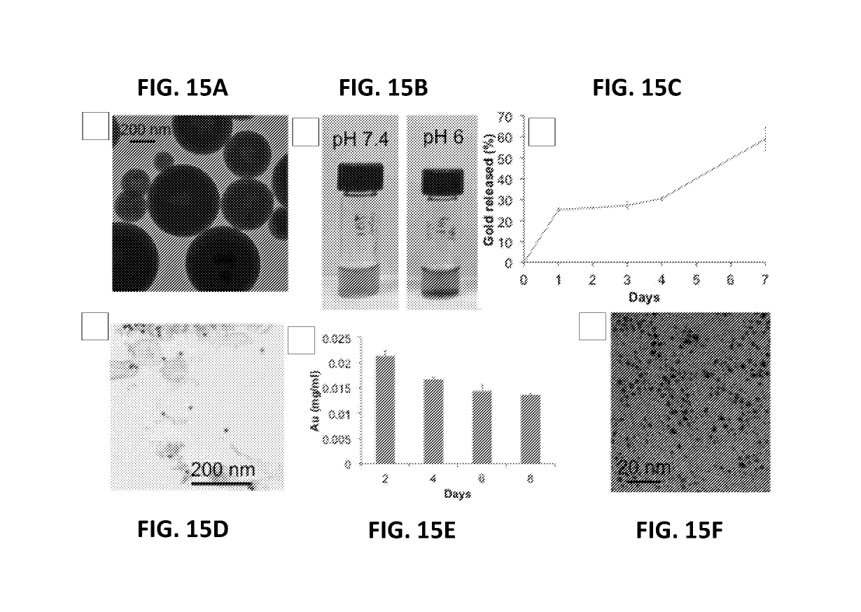

The stability in storage and degradability of Au-PCPP is demonstrated in FIGS. 15A-15F. GSAN loaded PCPP-NP should behave in a very similar fashion due to the strong chemical similarity between AuNP and GSAN. TEM on AuPCPP six months after synthesis and stored at 4° C. shows the stability of these nanoparticles out of the body (FIG. 15A). The nanoparticles are stable in PBS at pH 7.4 (the pH of blood), whereas at pH 6 (the pH of endosomes), the nanoparticles dissociated, as evidenced by the red color of free gold nanoparticles in solution (FIG. 15B). Gold core release was evaluated via incubating Au-PCPP in 10% serum in PBS at 37° C. for 7 days and sampling the liquid periodically. The samples were centrifuged to exclude Au-PCPP and the released gold cores in the supernatant evaluated with ICP-MS (FIG. 15C). 59% release within 7 days was observed, providing strong evidence for the biodegradability of AuPCPP. TEM on cells incubated with AuPCPP revealed that the aggregates break down in vitro, as dispersed gold cores were observed within cells (FIG. 15D). When macrophages were incubated with AuPCPP for 24 hours, then incubated with fresh media, we observed release of the gold into the media over time, indicating that the AuPCPP will be excreted from reticuloendothelial system (RES) cells (FIG. 15E). 66% of the gold that had been taken up was released over 8 days. TEM images of AuNP released from Au-PCPP after degradation of PCPP in a mildly acidic buffer were acquired. These AuNP were the same size as AuNP before incorporation into PCPP (FIG. 15F).

### Gold Loaded and GSAN Loaded PCPP-NP Biocompatibility

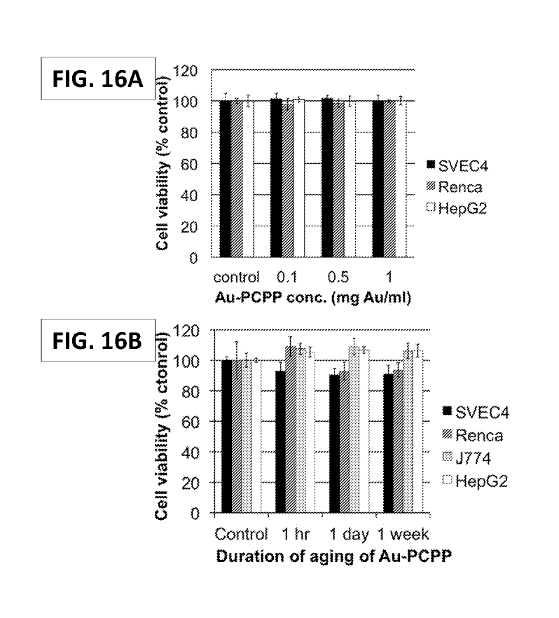

Preliminary data shows gold loaded PCPP-NP to be biocompatible even after incubation for 24 hours at 0.5 mg Au/ml with J774A.1 cells (FIG. 16A). No effect on HepG2 viability was seen at extended timepoints after incubation with AuPCPP for 8 hr (FIG. 16B). Additional toxicity screening will be done on PCPP-NP samples that are aged for one week in cell culture media, to test the toxicity of the breakdown products. For example, the results of such an experiment are shown where it can be seen that there is no effect of AuPCPP degradation products on HepG2 viability.

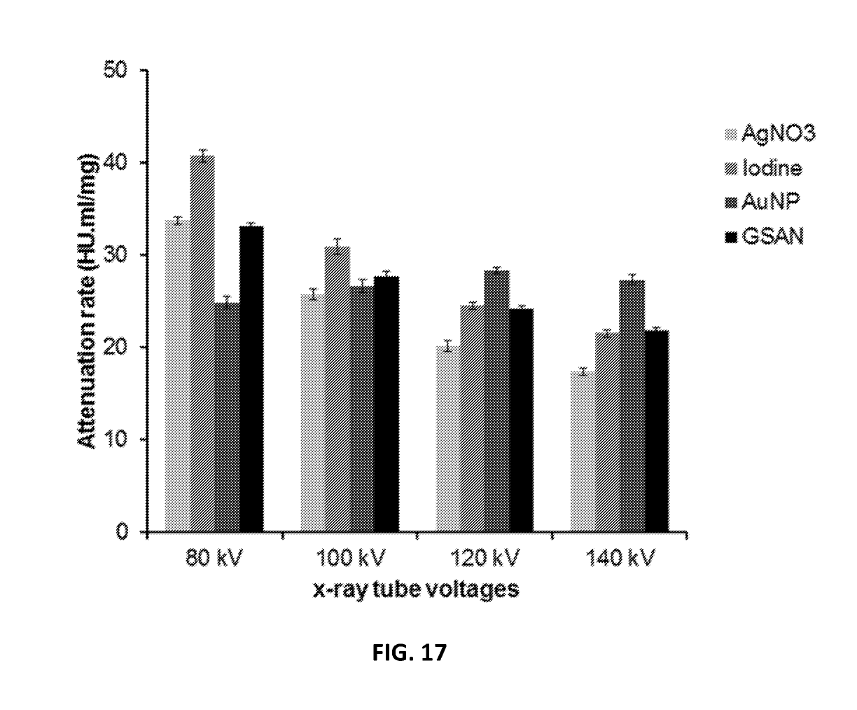

FIG. 17 is a graph depicting a comparison of various contrast agents according to attenuation rate in computed tomography and x-ray tube voltages according to aspects of the present invention.

### Example: Silver Sulfide Nanoparticles and Multimodal Nanoprobes

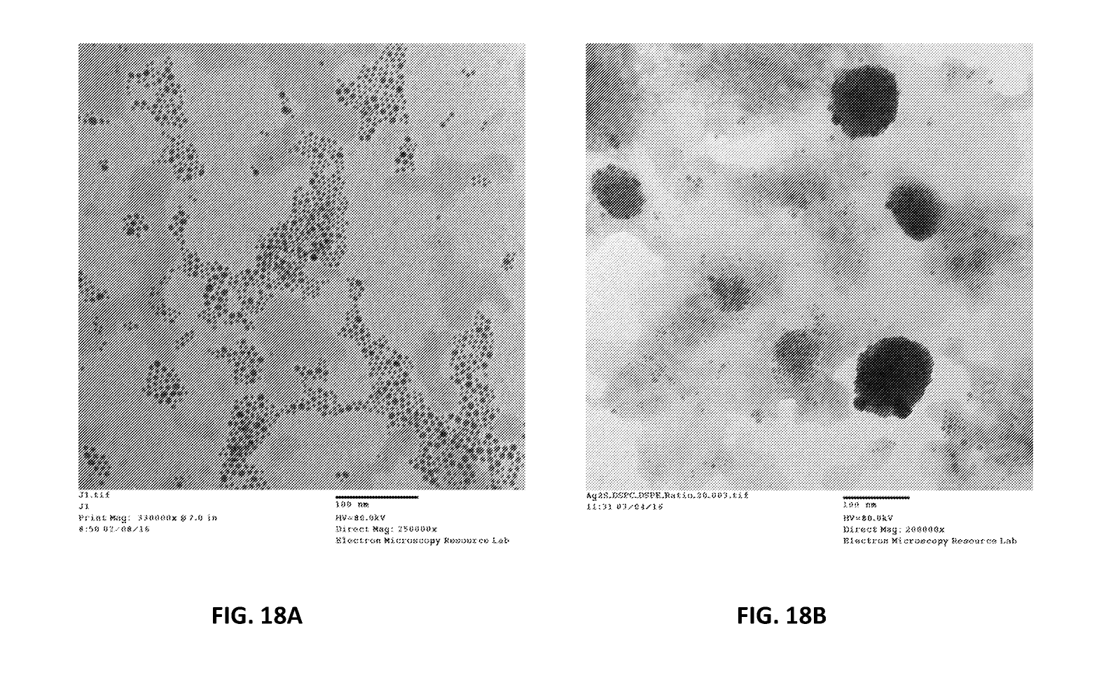

Silver chalcogenide nanoparticles (Ag₂X, where X=S, Se, or Te) have been reported to possess low toxicity and high biocompatibility, much like GSAN or AuNP. Additionally, silver sulfide (Ag₂S) nanoparticles have been successfully synthesized and shown to be suitable for contrast enhancement in X-ray imaging modalities (FIGS. 18A and 18B).

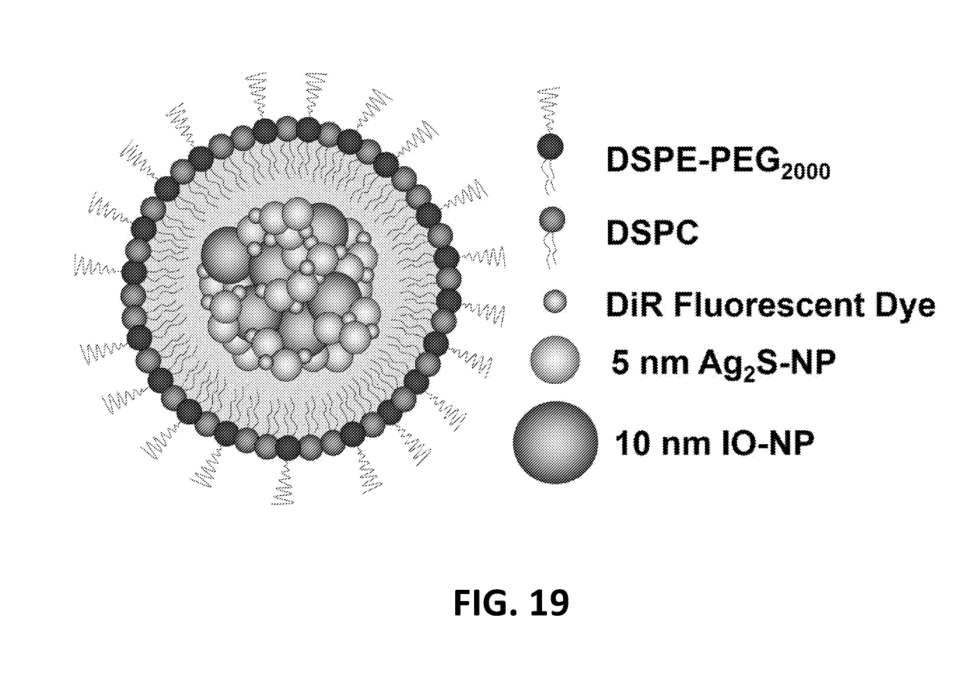

Moreover, it has surprisingly been found that a multimodal platform suitable for use as an imaging agent in DE mammography, CT scanning, MRI, and fluorescence optical imaging can be produced with Ag₂S nanoparticles (FIG. 19) in accordance with embodiments of the present invention. Such a multimodal platform provides versatility and efficiency, as only one imaging agent need be used with numerous scanning technologies. Ag₂S nanoparticles (“Ag₂S-NP”) approximately 5 nm in size are coated with dodecanethiol (DT). DT provides colloidal stability and facilitates control of the Ag₂S-NP size. Hydrophobic Ag₂S-NP with a core size of 5 nm were first synthesized via a thermal decomposition single-source precursor approach. In an exemplary synthesis, 0.1 mmol of (C₂H₅)₂NCS₂Ag was added into 10 g of 1-dodecanethiol (DT) in a 100 mL three-neck round-bottom flask at room temperature. The reaction mixture was degassed for 5 minutes under constant stirring and was then heated to 210° C. at a heating rate of 15° C./min and kept at 210° C. for 1 hour under N₂ atmosphere. The mixture was then cooled to room temperature and was washed three times with ethanol via centrifugation at 7000 rcf for 20 minutes. The as-prepared DT-coated Ag₂S-NP was dried under vacuum for the subsequent synthesis of multimodal nanoprobe.

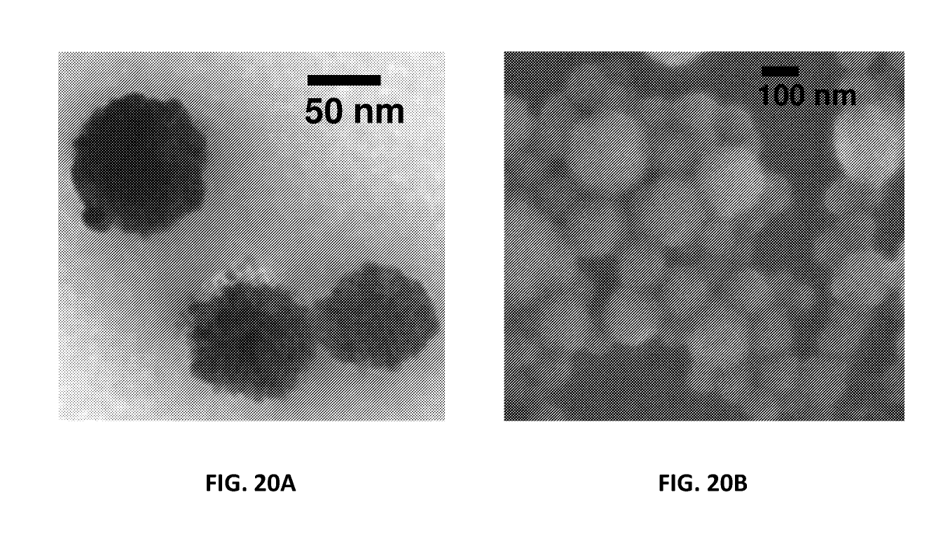

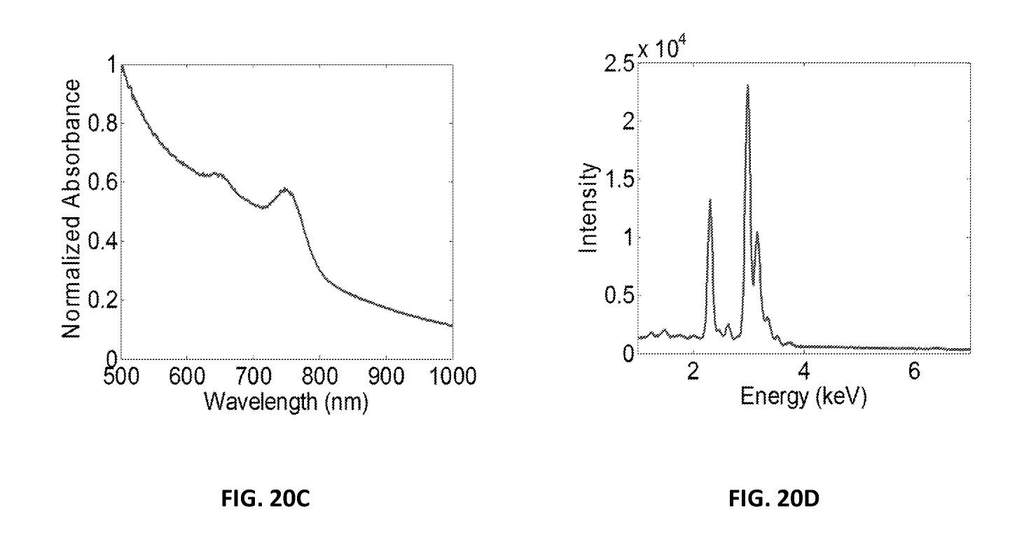

Iron oxide nanoparticles (“IO-NP”) approximately 10 nm in size are coated with oleic acid. Oleic acid plays a similar role for IO-NP as dodecanethiol for Ag₂S-NP. IO-NP are known for their use in MRI techniques. DiR near-infrared fluorescent dye, for optical contrast, is encapsulated with the DT-coated Ag₂S-NP and the oleic acid-coated IO-NP in micelles formed with a mixture of natural and polyethylene glycol-modified phospholipids (DSPC and DSPE-mPEG₂₀₀₀, respectively) to form a multimodal nanoprobe (FIGS. 20A and 20B). The phospholipids coat the nanocrystals, providing them with water solubility and biocompatibility. A combined solution (1000 μL) of Ag₂S-NP (20 mg), IO-NP (100 μg), DiR (100 μg), DSPC (1.1 mg) and DSPE-mPEG₂₀₀₀ (3.9 mg) was added directly to a glass vial containing 10 mL of DI water. The mixture was emulsified for approximately 20 seconds using a sonicator and was allowed to stir overnight to remove the chloroform. The resulting solution was centrifuged at 2630 rcf for 10 min to remove aggregates and was then filtered through a 0.22 μm filter to remove any oversized particles. Using 10 kDa MWCO tubes, the nanoparticles were concentrated and washed three times with DI water. Finally, all empty micelles and smaller sized particles were removed via potassium bromide density gradient centrifugation and the collected pellet was resuspended in PBS. The components are mixed at a mass ratio of 200 Ag₂S-NP:50 phospholipids:1 IO-NP:1 DiR to achieve adequate DE mammography and CT scanning enhancement from the Ag₂S-NP, while maintaining a sufficient level of IO-NP and DiR concentrations to produce MRI and optical fluorescence contrast, respectively.

UV-vis absorption spectroscopy of the multimodal nanoprobes (FIG. 20C) indicated a peak at 745 nm from the DiR, and the overall trend of the spectroscopy indicated agreement with observations of Ag₂S and iron oxide. Moreover, energy dispersive X-ray spectroscopy of the nanoprobes (FIG. 20D) confirmed the presence of Ag₂S and iron oxide.

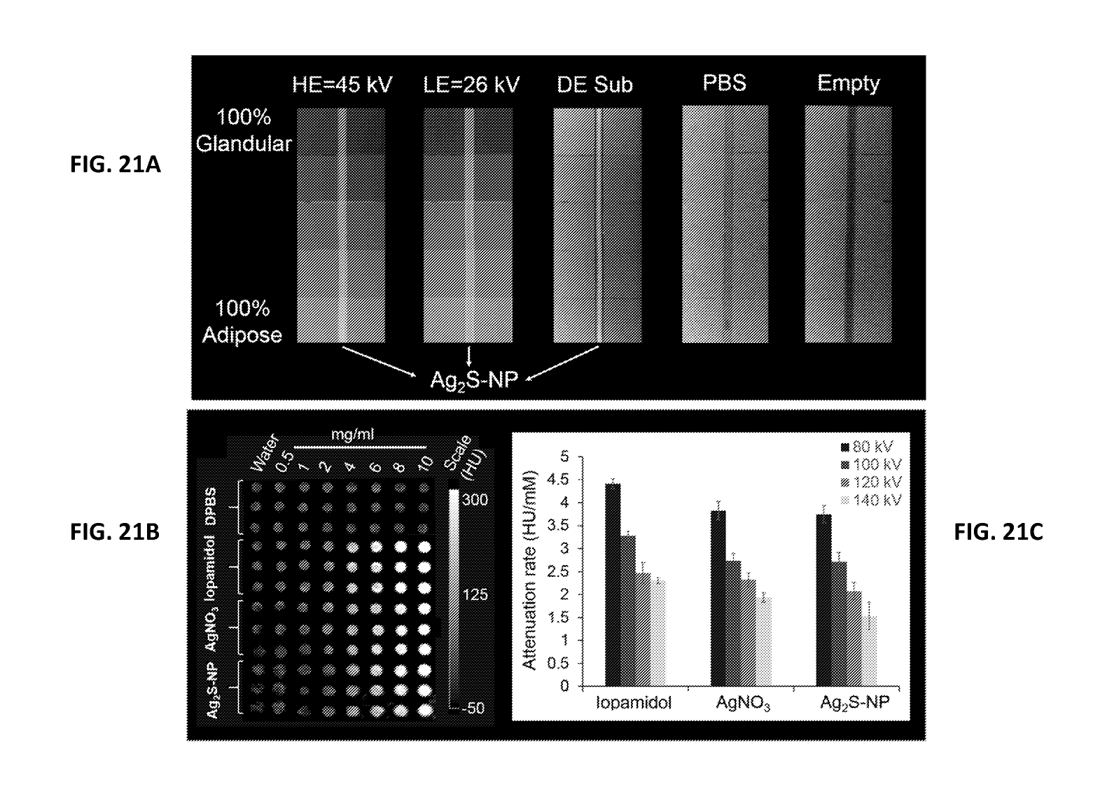

DE mammography images of the multimodal nanoprobe including Ag₂S-NP are presented in FIG. 21A. Images were acquired at low energy (LE) and high energy (HE). A weighted logarithmic subtraction was performed to obtain dual-energy (DE) images; it can be seen that Ag₂S provides excellent DE contrast in comparison to the PBS and empty controls. CT phantom imaging also showed comparable contrast between FDA-approved iodinated base contrast agent iopamidol, silver nitrate, and the Ag₂S-NP of the nanoprobes (FIGS. 21B and 21C).

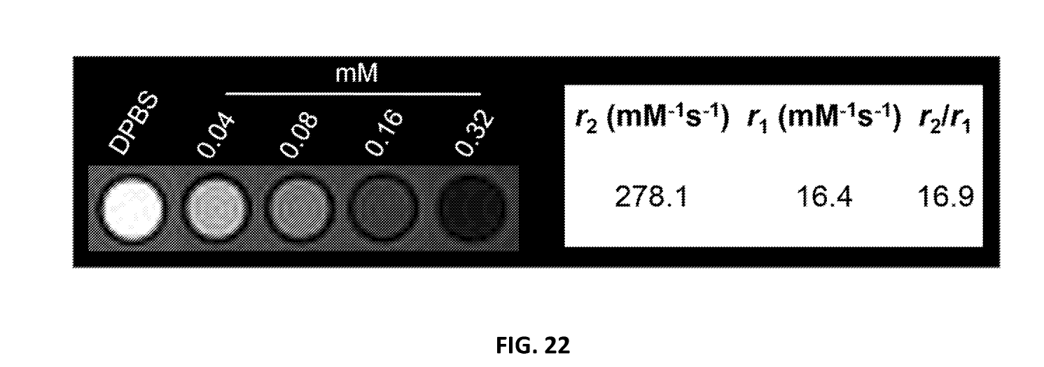

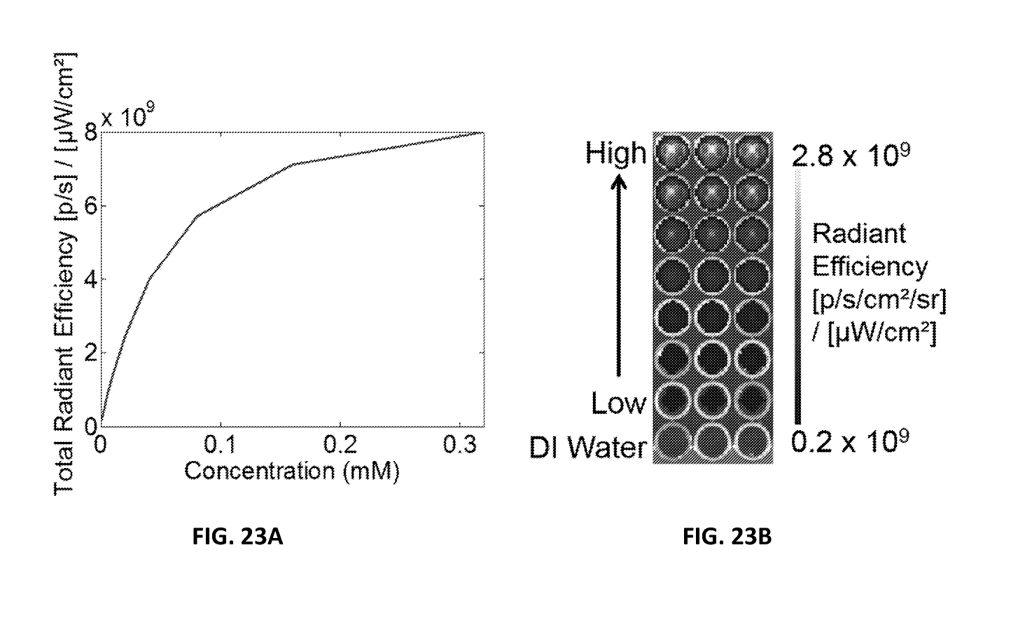

The nanoprobes also showed great potential for T₂-weighted imaging for MRI. MRI phantom imaging of the nanoprobes yielded a substantial r₂ value (FIG. 22). The phantom imaging of the fluorescence provided by the DiR near-infrared fluorescent dye also indicated great potential for optical imaging (FIGS. 23A and 23B).

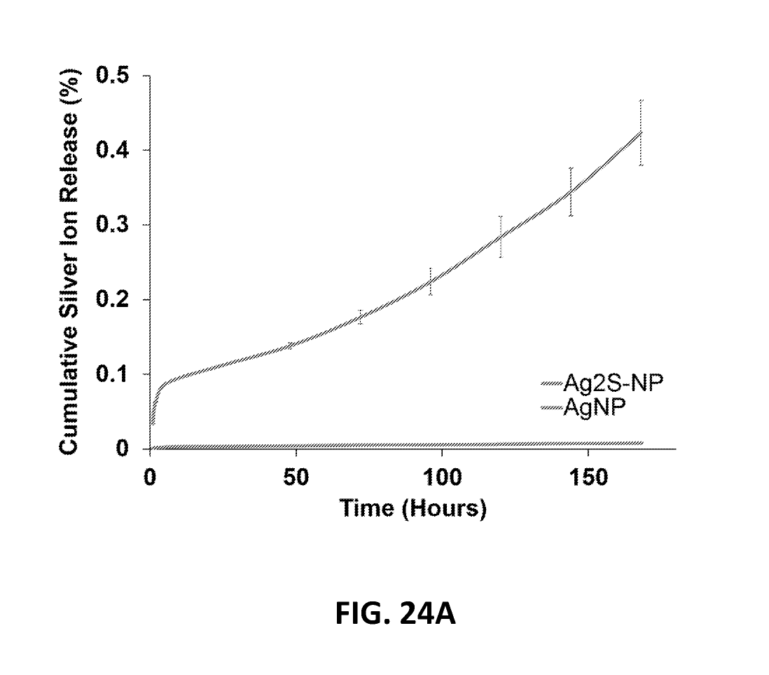

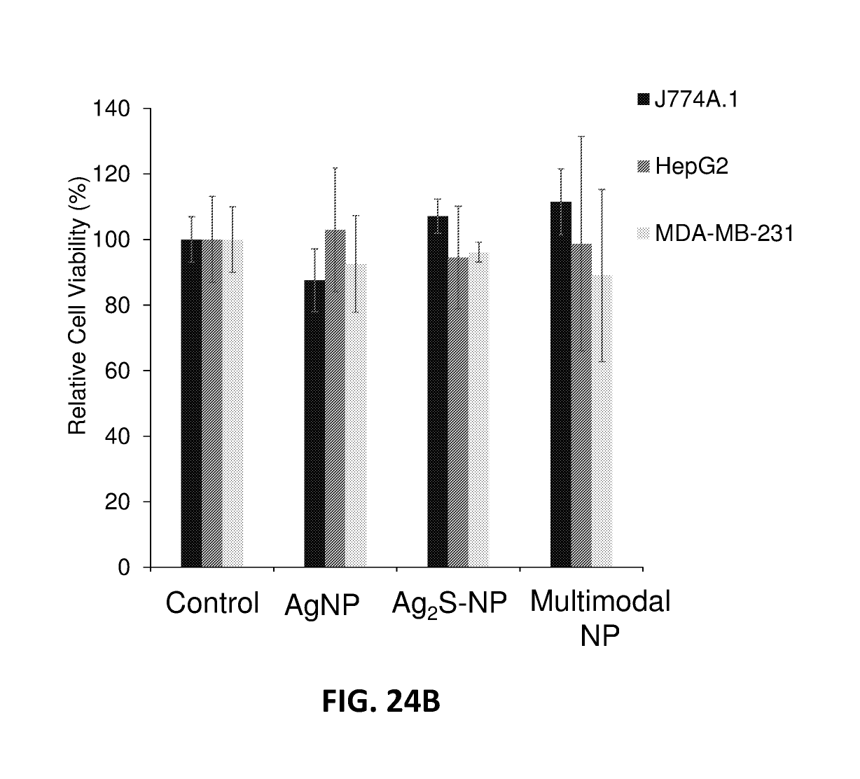

Additionally, biocompatibility studies indicated that there was minimal silver leaching from the Ag₂S-NP in comparison to AgNP (pure silver nanoparticles) (FIG. 24A). This was consistent with cell viability studies showing that Ag₂S-NP were biocompatible with three different cell lines (J774A.1, HepG2, and MDA-MB-231 cell lines) (FIG. 24B).

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. Nanoparticles, for use as a contrast agent for medical imaging techniques, each nanoparticle comprising:
i) a core including:
a contrast element, wherein the contrast element has a K-edge value ranging from about 17 to about 49 keV; and
a stabilizing element which minimizes at least one of cytotoxicity or immunoreactivity of the contrast element; and
ii) a first coating layer encapsulating the core, the first coating layer configured to render the nanoparticles soluble in a biological medium.

2. The nanoparticles of claim 1, further comprising a second coating layer configured to delay an in vivo release of the core.

3. The nanoparticles of claim 1, wherein the first coating layer is comprised of one or more first coating layer components selected from the group consisting of small molecules, peptides, sugars, lipids, proteins, and polymers.

4. The nanoparticles of claim 3, wherein the first coating layer is comprised of one or more first coating layer components selected from the group consisting of lipoic acid, oleic acid, glutathione, dodecanethiol, natural phospholipids, polyethylene glycol modified phospholipids, 11-mercapto-undecanoic acid, thioglucose, lecithin, dimyristoyl phosphatidylcholine, albumin, apolipoprotein AI thio-polyethylene glycol, polyacrylic acid, poly(D,L-lactic-co-glycolic acid), polycaprolactone, poly(vinyl-pyrrolidone), poly(acryl-amide), and poly(glycerol).

5. The nanoparticles of claim 2, wherein the second coating layer is comprised of one or more biodegradable carrier matrices.

6. The nanoparticles of claim 5, wherein the one or more biodegradable carrier matrices are selected from the group consisting of spermine and polyphosphazene.

7. The nanoparticles of claim 1, wherein the contrast element is selected from the group consisting of Zr, Nb, Mo, Tc, Ru, Rh, Pd, Ag, Cd, In, Sn, Sb, Te, I, Cs, Ba, La, Ce, Pr, Nd, Pm, Sm and Eu.

8. The nanoparticles of claim 1, wherein the stabilizing element is selected from the group consisting of B, C, N, O, F, Al, Si, P, S, Cl, Ti, V, Cr, Mn, Fe, Co, Ni, Cu, Zn, Ga, Ge, As, Se, Br, Y, Zr, Nb, Mo, Ru, Rh, Pd, Ag, Cd, In, Sn, Sb, Te, I, Ba, La, Ce, Pr, Nd, Pm, Sm, Eu, Gd, Tb, Dy, Ho, Er, Tm, Yb, Lu, Hf, Ta, W, Re, Os, Ir, Pt, Au and Bi.

9. The nanoparticles of claim 2, further comprising a third coating layer including at least one of fluorophores or targeting moieties.

10. The nanoparticles of claim 1, wherein the contrast element exists as a compound.

11. The nanoparticles of claim 1, wherein the stabilizing element exists as a compound.

12. The nanoparticles of claim 1, wherein the stabilizing element forms a shell around the contrast element.

13. The nanoparticles of claim 1, wherein the stabilizing element is alloyed with the contrast element.

14. The nanoparticles of claim 1, wherein the stabilizing element forms a compound with the contrast element.

15. The nanoparticles of claim 1, wherein the stabilizing element and the contrast element both contribute to a contrast generation.

16. The nanoparticles of claim 1, wherein the biological medium is serum.

17. The nanoparticles of claim 5, wherein the contrast element is Ag and the stabilizing element is Au.

18. The nanoparticles of claim 5, wherein the contrast element forms 1% to 99% by weight of the core.

19. The nanoparticles of claim 2, wherein the second coating layer encapsulates the first coating layer.

20. The nanoparticles of claim 14, wherein the contrast element is Ag and the stabilizing element is S.

21. The nanoparticles of claim 20, wherein the nanoparticles are encapsulated with iron oxide nanoparticles and the first coating layer includes dodecanethiol, oleic acid, natural phospholipids, and polyethylene glycol modified phospholipids.

22. A kit for contrast imaging comprising:
(i) a dry-powder dosage formulation including a contrast agent comprising a plurality of nanoparticles, the plurality of nanoparticles each having:
a core comprising:
a stabilizing element selected from the group consisting of B, C, N, O, F, Al, Si, P, S, Cl, Ti, V, Cr, Mn, Fe, Co, Ni, Cu, Zn, Ga, Ge, As, Se, Br, Y, Zr, Nb, Mo, Ru, Rh, Pd, Ag, Cd, In, Sn, Sb, Te, I, Ba, La, Ce, Pr, Nd, Pm, Sm, Eu, Gd, Tb, Dy, Ho, Er, Tm, Yb, Lu, Hf, Ta, W, Re, Os, Ir, Pt, Au and Bi; and
a contrast element, wherein the contrast element has a K-edge value ranging from about 17 to about 49 keV; and
a first coating layer encapsulating the core, the first coating layer configured to render the nanoparticles soluble in a biological medium.
(ii) a pharmaceutically acceptable carrier solution suitable for injection for reconstituting the dosage formulation.

23. A method for dual energy x-ray imaging, comprising the step of administering to a subject the nanoparticles of claim 1 as a contrast agent;
acquiring an image with a low energy spectrum; and
acquiring an image with a high energy spectrum.
